(19) United States
(12) Patent Application Publication
(10) Pub. No.: US 2010/0234341 A1
(43) Pub. Date: Sep. 16, 2010

(54) SUBSTITUTED PYRIMIDINES AS ADENOSINE RECEPTOR ANTAGONISTS

(76) Inventors: Marion Lanier, San Diego, CA (US); Deborah Slee, Cardiff, CA (US); Emily Lin, San Bruno, CA (US); Zhiyong Robert Luo, New City, NJ (US); Yongsheng Chen, San Diego, CA (US); Manisha Moorjani, San Diego, CA (US); Binh G. Vong, San Diego, CA (US); John Tellew, La Jolla, CA (US)

Correspondence Address: COOLEY LLP;ATTN: Patent Group, Suite 1100, 777 - 6th Street, NW, WASHINGTON, DC, 20001, US

(21) Appl. No.: 12/517,546

(22) PCT Filed: Dec. 4, 2007
(86) PCT No.: PCT/US07/86380

§ 371 Date: Apr. 13, 2010

Related U.S. Application Data
(60) Provisional application No. 60/868,517, filed on Dec. 4, 2006.

(51) Int. Cl. A61K 31/337; C07D 403/14; A61K 31/506; C07D 413/14; A61K 31/5377; C07D 205/06; A61P 9/10; A61P 1/00; A61P 13/12; A61P 3/10; A61P 11/06; A61P 25/28

(52) U.S. Cl. 514/210.2; 544/324; 514/275; 544/122; 514/235.8; 548/950

(57) ABSTRACT

Compounds of formula (I) including pharmaceutically acceptable salts, esters, solvates and stereoisomers thereof, R¹, R² and R³ are as defined herein. Pharmaceutical compositions containing a compound of structure (I), as well as methods relating to the use thereof, are also disclosed.

### CROSS-REFERENCE TO RELATED APPLICATION

This application claims the benefit of U.S. Provisional Application Ser. No. 60/868,517, filed Dec. 4, 2006, which application is incorporated by reference herein in its entirety.

### BACKGROUND OF THE INVENTION

1. Field of the Invention

The present invention relates to new antagonists of adenosine receptors, in particular antagonists of the A_{2A} adenosine receptor subtype, the use of said compounds in the treatment of diseases and disorders susceptible of being ameliorated by antagonism of adenosine receptors, and to pharmaceutical compositions comprising said compounds. Disorders of the central nervous system which are known to be improved by the use of antagonists of the A_{2A} adenosine receptors include, for example, Parkinson's disease, Huntington's disease, restless leg syndrome and dyskinesia.

2. Description of the Related Art

The effects of adenosine are mediated through at least four specific cell membrane receptors so far identified and classified as receptors A₁, A_{2A}, A_{2B} and A₃ belonging to the G protein-coupled receptor family. The A₁ and A₃ receptors down-regulate cellular cAMP levels through their coupling to G proteins, which inhibit adenylate cyclase. In contrast, A_{2A} and A_{2B} receptors couple to G proteins that activate adenylate cyclase and increase intracellular levels of cAMP. Through these receptors, adenosine regulates a wide range of physiological functions.

Thus, in the cardiovascular system the activation of the A₁ receptor protects cardiac tissue from the effects of ischemia and hypoxia. A similar protective effect is also produced by antagonism of the A_{2A} receptor, which enhances Aᵢ-receptor-induced antiadrenergic responses and may also be useful in the treatment of acute myocardial ischemia and supraventricular arrhythmias (Norton G R et al. Am J. Physiol. 1999; 276(2 Pt 2):H341-9; Auchampach J A, Bolli R. Am J. Physiol. 1999; 276(3 Pt 2):H1113-6). In addition, the A_{2B} adenosine receptor subtype (Feoktistov, I. et al., Pharmacol. Rev. 1997, 49, 381-402) appears to be involved in the control of vascular tone and the regulation of vascular smooth muscle growth.

In the kidney, adenosine exerts a biphasic action, inducing vasodilation at high concentrations and vasoconstriction at low concentrations. Thus, adenosine plays a role in the pathogenesis of some forms of acute renal failure that may be ameliorated by A₁ receptor antagonists (Costello-Boerrigter L C, et al. Med Clin North Am. 2003 March; 87(2): 475-91; Gottlieb S S., Drugs. 2001; 61(10): 1387-93).

Adenosine is also involved in the physiopathology of the immune system. It can induce degranulation of activated human mast cells through the A_{2B} and/or A₃ receptor. Thus A_{2B} and/or A₃ antagonists prevent mast cell degranulation and are, therefore, useful in the treatment, prevention or suppression of disease states induced by activation of the A_{2B} and/or A₃ receptor and mast cell degranulation. These disease states include but are not limited to asthma, myocardial reperfusion injury, allergic reactions including but not limited to rhinitis, urticaria, scleroderm arthritis, other autoimmune diseases and inflammatory bowel diseases.

Furthermore, in the respiratory system adenosine induces bronchoconstriction, modulates airway inflammation and promotes neutrophil chemotaxis. Therefore, an adenosine antagonist would be particularly useful in the treatment of asthma.

In the gastrointestinal and metabolic system, the A_{2B} adenosine receptor subtype (Feoktistov, I. et al., Pharmacol. Rev. 1997, 49, 381-402) seems to be involved in the regulation of hepatic glucose production, the modulation of intestinal tone, as well as intestinal secretion. Thus, A_{2B} antagonists may also be useful in the treatment of diabetes mellitus and obesity.

In the central nervous system adenosine is a potent endogenous neuromodulator, which controls the presynaptic release of many neurotransmitters and is thus involved in motor function, sleep, anxiety, pain and psychomotor activity. All adenosine receptor subtypes are present in the brain, with A₁ and A_{2A} subtypes being differentially distributed. The former are found predominantly in the hippocampus and cortex, whilst the latter are found mainly in the striatum. Adenosine A_{2A} receptors modulate the release of GABA in the striatum, which possibly regulates the activity of medium spiny neurons.

Thus, A_{2A} receptor antagonists may be a useful treatment for neurodegenerative movement disorders such as Parkinson and Huntington's disease (Tuite P, et al., J. Expert Opin Investig Drugs. 2003; 12: 1335-52; Popoli P. et al. J. Neurosci. 2002; 22:1967-75), dystonias such as restless leg syndrome (Happe S, et al., Neuropsychobiology. 2003; 48: 82-6), and dyskinesias such as those caused by prolonged use of neuroleptic and dopaminergic drugs (Jenner P. J Neurol. 2000; 247 Suppl2: II43-50).

In the treatment of Parkinson's disease an A_{2A} antagonist may be useful not only as monotherapy, but also when administered in combination with L-DOPA and/or one or more of the following drugs: dopamine agonists, inhibitors of dopamine decarboxylase, catechol-O-methyltransferase inhibitors and inhibitors of monoamine oxidase.

In addition, A_{2A} antagonists may have therapeutic potential as neuroprotectants (Stone T W. et al., Drug. Dev. Res. 2001; 52: 323-330), and in the treatment of sleep disorders (Dunwiddie T V et al., Ann. Rev. Neurosci. 2001; 24: 31-55).

It has now been found that certain 4-aminopyrimidine derivatives are novel potent antagonists of the A_{2A} adenosine receptor and can therefore be used in the treatment or prevention of diseases susceptible to amelioration by antagonism of the adenosine receptor.

Further objectives of the present invention are to provide a method for preparing said compounds; pharmaceutical compositions comprising an effective amount of said compounds; the use of the compounds in the manufacture of a medicament for the treatment of pathological conditions or diseases susceptible of being improved by antagonism of an adenosine receptor, in particular by antagonism of the A_{2A} adenosine receptor; methods of treatment of pathological conditions or diseases susceptible to amelioration by antagonism of an adenosine receptor, in particular by antagonism of the A_{2A} adenosine receptor comprising the administration of the compounds of the invention to a subject in need of treatment and combinations of said compounds with one or more of the following drugs: L-DOPA, dopamine agonists, inhibitors of dopamine decarboxylase, catechol-O-methyltransferase inhibitors and inhibitors of monoamine oxidase.

### BRIEF SUMMARY OF THE INVENTION

In brief, this invention is generally directed to adenosine receptor antagonists, as well as to methods for their preparation and use, and to pharmaceutical compositions containing the same. More specifically, the adenosine receptor antagonists of this invention are compounds having the following general structure (I):

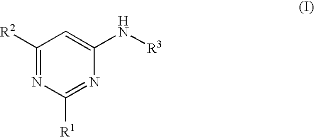

and pharmaceutically acceptable salts, esters, solvates and stereoisomers thereof, wherein R¹, R² and R³ are as defined below.

The compounds of this invention may generally be used to treat a variety of disorders or conditions, particularly those which benefit from inhibition of adenosine (particularly A_{2A}) receptors. Accordingly, in another embodiment, methods are disclosed for treating one or more of a variety of diseases or conditions, including (but not limited to) ischemia, supraventricular arrhythmias, acute renal failure, myocardial reperfusion injury, autoimmune disease, inflammatory bowel diseases, asthma, diabetes mellitus, obesity, Parkinson disease, Huntington's disease, dystonia and dyskinesia.

The methods of this invention generally involve administering an effective amount of one or more compounds of this invention, typically in the form of a pharmaceutical composition, to an animal (also referred to here as a “patient”, including a human) in need thereof. Accordingly, in still another embodiment, compositions are disclosed containing one or more compounds of this invention and a pharmaceutically acceptable carrier and/or diluent.

These and other aspects of the invention will be apparent upon reference to the following detailed description. To that end, various references are set forth herein which describe in more detail certain procedures, compounds and/or compositions, and are hereby incorporated by reference in their entirety.

### DETAILED DESCRIPTION OF THE INVENTION

As mentioned above, the present invention is directed generally to compounds useful as adenosine receptor antagonists. The compounds of this invention have the following structure (I):

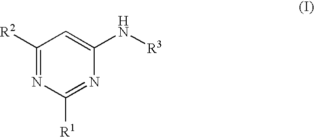

and pharmaceutically acceptable salts, esters, solvates and stereoisomers thereof, wherein:
R¹ is a heterocycle optionally substituted by one or more members selected from the group of lower alkyl, lower alkoxy, halogen and cyano;
R² is NR⁴R⁵ or a heterocycle, wherein the heterocycle is substituted by 0 to 4 R⁴ groups;
R³ is H, R⁶, OR⁶, COR^{E}, CONR⁶R⁷, COOR⁶, or a heteroaryl having at least one nitrogen wherein the heteroaryl is optionally substituted by 0 to 4 R⁴;

R⁴ is at each occurrence selected from the group of lower alkyl, lower alkoxy, alkoxyalkyl, oxo, cyano, halogen, hydroxy, —C(O)-alkyl, lower alkenyl, aryl, heteroaryl, arylalkyl, heteroarylalkyl, heterocycle and heterocyclealkyl, wherein the lower alkyl, lower alkoxy, alkoxyalkyl, aryl, heteroaryl, arylalkyl, heteroarylalkyl, heterocycle and heterocyclealkyl groups are optionally substituted by one or more lower alkyl, halogen, lower alkoxy, hydroxyl, cyano, aryl and —C(O)-alkyl;

R⁵ is at each occurrence selected from the group of hydrogen, lower alkyl, lower alkoxy and alkoxyalkyl;
R⁶ is lower alkyl, arylalkyl, heteroaryl or heterocyclealkyl, wherein the lower alkyl, arylalkyl, heteroaryl and heterocyclealkyl groups are optionally substituted by one or more members selected from the group of lower alkyl, lower alkoxy, hydroxyl, oxo, halogen, amino, alkylamino and dialkylamino; and
R⁷ is hydrogen or lower alkyl, wherein the lower alkyl group is optionally substituted by one or more members selected from the group of alkoxy, hydroxyl, oxo, halogen, amino, alkylamino and dialkylamino.

Other aspects of the present invention are: a) pharmaceutical compositions containing a pharmaceutically effective amount of a compound of the invention, b) the use of a compound of the invention in the manufacture of a medicament for the treatment of diseases susceptible of being improved by antagonism of an adenosine receptor, in particular by antagonism of the A_{2A} adenosine receptor; and c) methods of treatment of diseases susceptible to amelioration by antagonism of an adenosine receptor, in particular by antagonism of the A_{2A} adenosine receptor, which methods comprise the administration of the compounds of the invention to a subject in need of treatment. The present invention also includes administration, to a subject in need thereof, of a compound of the invention in combination with one or more of the following drugs: L-DOPA, dopamine agonists, inhibitors of dopamine decarboxylase, catechol-β-methyltransferase inhibitors and inhibitors of monoamine oxidase.

As used herein the term lower alkyl embraces optionally substituted, linear or branched alkyl radicals having 1 to 8 carbon atoms. Typically lower alkyl groups have 1 to 6 or 1 to 4 carbon atoms. Typical examples of substituents in said alkyl groups are halogen, hydroxy and amino.

Examples of lower alkyl groups include methyl, ethyl, n-propyl, i-propyl, n-butyl, sec-butyl and tert-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl and iso-hexyl radicals.

As used herein, the term lower alkoxy embraces optionally substituted, linear or brached oxy-containing radicals each having alkyl portions of 1 to 8, typically 1 to 6 and more typically 1 to 4 carbon atoms. Typical examples of substituents in said alkoxy groups are halogen, hydroxy and amino.

Examples of lower alkoxy groups include methoxy, ethoxy, n-propoxy, i-propoxy, n-butoxy, sec-butoxy, t-butoxy, trifluoromethoxy, difluoromethoxy, hydroxymethoxy, 2-hydroxyethoxy or 2-hydroxypropoxy.

As used herein, the term lower alkylthio embraces radicals containing an optionally substituted, linear or brached alkyl radicals of 1 to 8, typically 1 to 6 and more typically 1 to 4 carbon atoms. Typical examples of substituents in said alkoxy groups are halogen, hydroxy and amino.

Examples of optionally substituted lower alkylthio radicals include methylthio, ethylthio, n-propylthio, i-propylthio, n-butylthio, sec-butylthio, t-butylthio, trifluoromethylthio, difluoromethylthio, hydroxymethylthio, 2-hydroxyethylthio or 2-hydroxypropylthio.

As used herein the term “acyl” refers to groups represented by the formula alkyl-C(═O)—, where the alkyl group may be substituted or unsubstituted.

As used herein, the term cyclic group embraces, unless otherwise specified, carbocyclic and heterocyclic radicals. The cyclic radicals can contain one or more rings. Carbocyclic radicals may be aromatic or alicyclic, for example cycloalkyl radicals. Heterocyclic radicals also include heteroaryl radicals.

As used herein, the term aromatic group embraces typically a 5- to 14-membered aromatic ring system, such as a 5- or 6-membered ring which may contain one or more heteroatoms selected from O, S and N. When no heteroatoms are present the radical is named aryl radical and when at least one heteroatom is present it is named heteroaryl radical. The aromatic radical can be monocyclic or polycyclic, such as phenyl or naphthyl. When an aromatic radical or moiety carries 2 or more substituents, the substituents may be the same or different.

As used herein, the term aryl radical embraces typically a C₅-C₁₄ monocyclic or polycyclic aryl radical such as phenyl, naphthyl, anthranyl or phenanthryl. When an aryl radical carries 2 or more substituents, the substituents may be the same or different.

As used herein, the term heteroaryl radical embraces typically a 5- to 14-membered ring system comprising at least one heteroaromatic ring and containing at least one heteroatom selected from O, S and N. A heteroaryl radical may be a single ring or two or more fused rings wherein at least one ring contains a heteroatom.

Examples of heteroaryls include pyridyl, pyrazinyl, pyrimidinyl, pyridazinyl, furyl, oxadiazolyl, oxazolyl, isoxazolyl, imidazolyl, thiazolyl, thiadiazolyl, thienyl, pyrrolyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl and pyrazolyl. When a heteroaryl radical carries 2 or more substituents, the substituents may be the same or different.

As used herein, the term heterocycle radical embraces typically a 5- to 14-membered ring system comprising at least one heterocyclic ring and containing at least one heteroatom selected from O, S and N. A heterocycle radical may be a single ring or two or more fused rings wherein at least one ring contains a heteroatom. A heterocycle radical may be aromatic, in which case it is a heteroaryl radical, or it may be non-aromatic.

Examples of aromatic heterocycles (i.e., heteroaryls) are provided above. Examples of non-aromatic heterocycles include piperidinyl, piperazinyl, morpholinyl, pyrrolidinyl, thiomorpholinyl, oxazolidinyl, imidazolidinyl, thiazolidinyl, azepanyl, [1,4]diazepanyl, [1,4]oxazepanyl and thiazepanyl.

As used herein, the term cycloalkyl embraces saturated optionally substituted carbocyclic radicals and, unless otherwise specified, a cycloalkyl radical typically has from 3 to 7 carbon atoms. The preferred substituents in said cycloalkyl groups are selected from halogen atoms, hydroxy groups, alkyl groups and amino groups.

Examples include cyclopropyl, cyclobutyl, cyclopentyl, cyclohexyl and cycloheptyl. It is preferably cyclopropyl, cyclopentyl or cyclohexyl. When a cycloalkyl radical carries 2 or more substituents, the substituents may be the same or different.

As used herein, some of the atoms, radicals, moieties, chains or cycles present in the general structures of the invention are “optionally substituted”. This means that these atoms, radicals, moieties, chains or cycles can be either unsubstituted or substituted in any position by one or more, for example 1, 2, 3 or 4, substituents, whereby the hydrogen atoms bound to the unsubstituted atoms, radicals, moieties, chains or cycles are replaced by chemically acceptable atoms, radicals, moieties, chains or cycles. When two or more substituents are present, each substituent may be the same or different.

The substituents of an “optionally substituted” structure may include, without limitation, one or more, typically one to four, and more typically one to two of the following substituents: alkyl, alkenyl, alkynyl, aryl, heteroaryl, alkoxy, aryloxy, alkylthio, arylthio, cycloalkyl, arylalkyl, amino, alkylamino, dialkylamino, amido (e.g. CONH2, CONHalkyl and CONHdialkyl and reverse NCOH or NCOalkyl), F, Cl, Br, I, CN, NO₂, NH₂, NHCH₃, NHCH₂CH₃, N(CH₃)₂, N(CH₂CH₃)₂, SH, SCH₃, OH, OCH₃, OCF₃, CH₃, and CF₃.

As used herein, the term halogen atom embraces chlorine, fluorine, bromine or iodine atoms typically a fluorine, chlorine or bromine atom, most preferably chlorine or fluorine. The term halo when used as a prefix has the same meaning

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X⁻) is associated with the positive charge of the N atom. X⁻ may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X⁻ is preferably an anion selected from chloride, bromide, iodide, sulphate, nitrate, acetate, maleate, oxalate, succinate or trifluoroacetate. More preferably X⁻ is chloride, bromide, trifluoroacetate or methanesulphonate.

As used herein, an N-oxide is formed from the tertiary basic amines or imines present in the molecule, using a convenient oxidising agent.

In one embodiment of the present invention in the compounds of formula (I), R¹ represents a heterocycle optionally substituted by one or more members selected from the group of lower alkyl, lower alkoxy, halogen and cyano.

According to one embodiment of the present invention, R¹ represents a heteroaryl group selected from the group of pyridinyl, furanyl, thiophenyl, thiazolyl, oxazole pyrazolyl, triazolyl, imidiazolyl, oxazolyl, isoxazolyl and oxadiazolyl groups which are optionally substituted by one or more substituents selected from the group of halogen, hydroxyl, amino, alkylamino, optionally substituted lower alkoxy and optionally substituted lower alkyl.

In one embodiment of the present invention R¹ represents a heteroaryl group selected from the following:

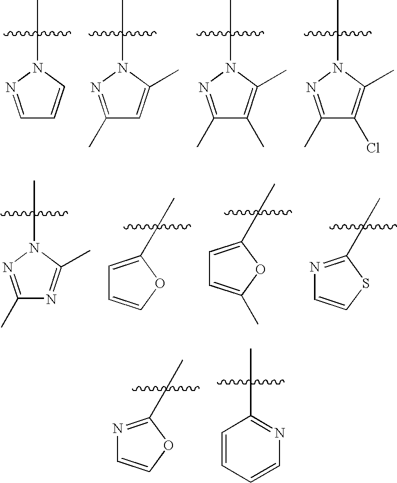

In one embodiment of the present invention in the compounds of formula (I), R² represents a heterocycle optionally substituted by 0 to 4 R⁴ groups.

In one embodiment of the present invention where R² is a heterocycle, R² represents pyrrolidinyl optionally substituted by one or more substituents selected from the group of alkyl, alkoxy, alkoxyalkyl, benzyloxy, phenoxyalkyl, hydroxyl, hydroxyalkyl, halogen, amino, alkylamino, dialkylamino, amido, —C(O)O-alkyl and morpholinyl.

In another embodiment of the present invention where R² is a heterocycle, R² represents piperidinyl optionally substituted by one or more substituents selected from the group of alkyl, alkoxy, alkoxyalkyl, benzyloxy, phenoxyalkyl, hydroxyl, hydroxyalkyl, halogen, amino, alkylamino, dialkylamino, amido, —C(O)O-alkyl and morpholinyl.

In another embodiment of the present invention where R² is a heterocycle, R² represents indolyl or isoindolyl optionally substituted by one or more substituents selected from the group of alkyl, alkoxy, alkoxyalkyl and cyano.

In another embodiment of the present invention where R² is a heterocycle, R² represents a monocyclic or bicyclic lactone optionally substituted by one or more alkyl or cycloalkyl groups.

In another embodiment of the present invention where R² is NR⁴R⁵, R² represents an N-alkoxyamino, anilinyl or aminopyridinyl optionally substituted with one or more substituents selected from the group of alkoxy and halogen, lactamyl, tetrahydropyridinyl optionally substituted by phenyl, piperazinyl optionally substituted by phenyl, benzyl or pyridinyl, azeridinyl, morpholinyl optionally substituted with one or more alkyl, alkylamino optionally substituted by one or more substituents selected from the group of alkoxy, hydroxyl, heterocyclyl, aryl and heteroaryl, and dialkylamino optionally substituted by one or more substituents selected from the group of alkoxy, hydroxyl, heterocyclyl, aryl and heteroaryl.

According to still another embodiment of the present invention in the compounds of formula (I), R² represents a heterocycle having at least one nitrogen atom, wherein the heterocycle is optionally substituted by one or more lower alkyl groups. Such hetereocycles include, for example, optionally substituted piperidinyl, piperazinyl, morpholinyl, thiomorpholinyl, pyrrolidinyl, isoquinolinyl, diazepanyl, dihydropyrrolyl, azepanyl, oxazepanyl, and pyrrolopyrazinyl.

According to still another embodiment of the present invention in the compounds of formula (I), R³ represents a hydrogen, acyl, heterocyclealkyl, arylalkyl, alkoxyl, alkyloxycorboxyl, dialkylamido, alkylamido or heteroaryl.

In one embodiment of the present invention R³ represents a group selected from the following:

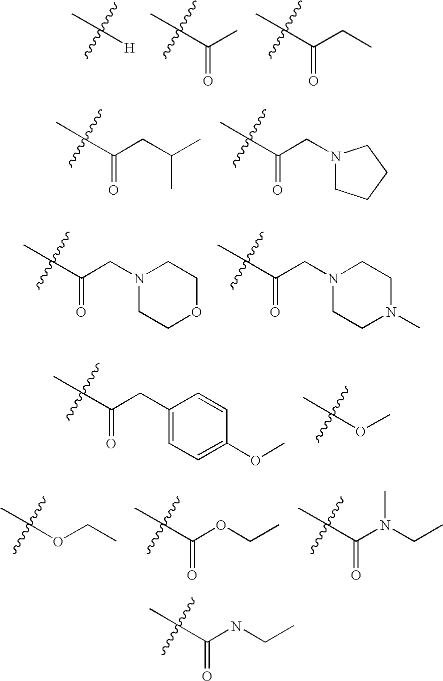

In another embodiment of the present invention R³ represents a group selected from the following:

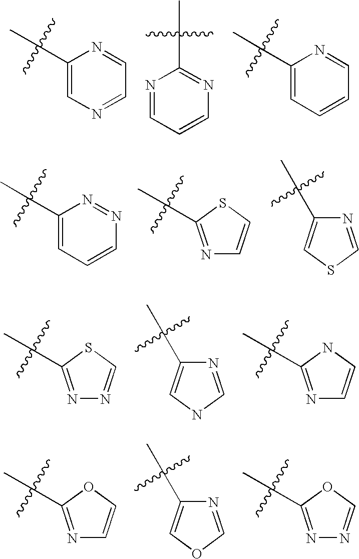

The compounds of the present invention may be prepared by one of the processes described below.

Compounds of formula (I) and in particular those of formulas (VIII) or (IX) where R¹ is a monocyclic or polycyclic heteroaryl group linked to the pyrimidine ring through a carbon atom and R² is a acyclic, monocyclic or polycyclic heteroaryl group linked to the pyrimidine ring through a nitrogen atom can be obtained as shown is Scheme 1.

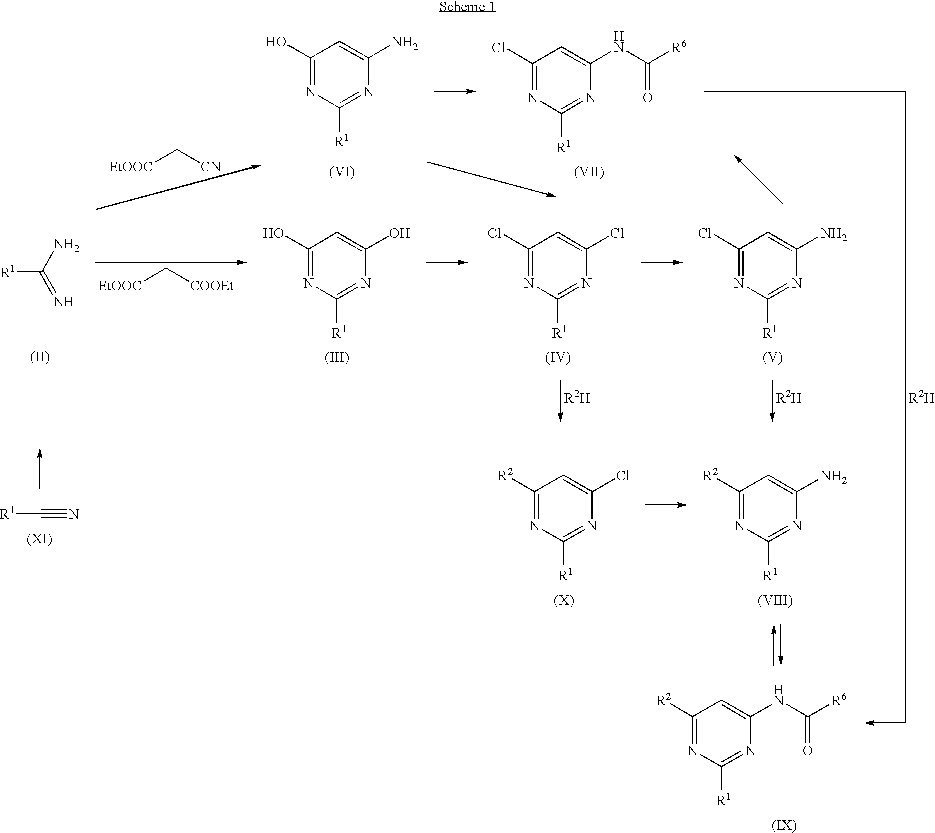

The carboxyamidines of formula (II), wherein R¹ is a monocyclic or polycyclic heteroaryl group linked to the carboxyamidine group through a carbon atom can be obtained by reacting a nitrile of formula (XI) with trimethylaluminum and ammonium chloride, in a solvent such as benzene, toluene or xylene, at a temperature from 80° C. to 120° C. It also can be obtained by reaction of a nitrile of formula (XI) with sodium methoxide in methanol at room temperature, followed by reaction with ammonium chloride at the same temperature.

The carboxyamidines of formula (II) can be reacted with diethyl malonate in a solvent such as methanol, ethanol, isopropyl alcohol, butyl alcohol or tetrahydrofuran, in the presence of a base, such as sodium methoxide, sodium ethoxide or potassium tertbutoxide and at a temperature from room temperature to the boiling point of the solvent to yield the pyrimidine-4,6-diols of formula (III).

The resulting pyrimidine-4,6-diols of formula (III) can be reacted with a chlorinated agent such a phosphorus oxychloride, phosphorus pentachloride or a mixture of them, in a solvent such as phosphorus oxychloride, benzene or toluene, at a temperature from room temperature to the boiling point of the solvent to yield the 4,6-dichloropyrimidine compounds of formula (IV). Optionally, the presence of a base such as dimethylaminoaniline, triethylamine or diisopropyl-ethylamine may be needed in this reaction step.

The reaction of the 4,6-dichloropyrimidine compounds of formula (IV) with ammonium hydroxide in a solvent such as methanol, ethanol, isopropyl alcohol or tetrahydrofuran, at a temperature from 80° C. to 140° C. produces the 6-chloropyrimidin-4-amines of formula (V).

The resulting the 6-chloropyrimidin-4-amines of formula (V) are reacted with a compound of formula R²—H wherein R² is an acyclic, monocyclic or polycyclic group linked to the pyrimidine ring through a nitrogen atom to yield the compounds of formula (VIII) which is a particular case of the compounds of formula (I) according to the invention. The reaction is carried out in a solvent such as dioxane, dimethylformamide, dimethylacetamide or dimethylsulfoxide, in the presence of a base, such as sodium hydride, potassium carbonate or cesium carbonate, at a temperature from 60° C. to 140° C.

The compounds of formula (VIII) can be acylated by an acid chloride and a base, such as pyridine, triethylamine or diisopropylethylamine, in a solvent such as tetrahydrofuran, methylene chloride, chloroform or pyridine, at a temperature from room temperature to the boiling point of the solvent to yield the compounds of formula (IX) which is a particular case of the compounds of formula (I) according to the invention. Compounds of formula (IX) can also be prepared by reaction of amine (VIII) with an anhydride, at a temperature from 80° C. to 160° C.

The 4,6-dichloropyrimidine compounds of formula (IV) can also be converted into the 4-chloropyrimidines of formula (X) by reaction with a compound of formula R²—H wherein R² is an acyclic, monocyclic or polycyclic group linked to the pyrimidine ring through a nitrogen atom. The reaction is carried out in a solvent such as dimethylformamide, dimethylacetamide or dimethylsulfoxide, in the presence of a base, such as sodium hydride, potassium carbonate or cesium carbonate, at a temperature from 60° C. to 140° C.

The resulting 4-chloropyrimidines of formula (X) can then be converted to the compounds of formula (VIII) according to the invention by reaction with ammonium hydroxide in a solvent such as methanol, ethanol, isopropyl alcohol or tetrahydrofuran, at a temperature from 80° C. to 140° C.

Alternatively, the compounds of formula (VIII) according to the invention can also be obtained from the compounds of formula (IX) by reaction with a mineral acid, such as hydrochloric acid or sulphuric acid, in a solvent such as water, methanol, ethanol or isopropyl alcohol, at a temperature from room temperature to the boiling point of the solvent.

The compounds of formula (IX) according to the invention can be obtained by reaction of the compounds of formula (VII) with compounds of formula R²H wherein R² is as hereinabove-defined. The reaction is carried out in a solvent such as dioxane, dimethylformamide, dimethylacetamide or dimethylsulfoxide, in the presence of a base, such as sodium hydride, potassium carbonate or cesium carbonate, at a temperature from 60° C. to 140° C.

The compounds of formula (VII) can be obtained from the 6-aminopyrimidin-4-ol compounds of formula (VI) by reaction with a carboxylic acid of formula R³COOH, wherein R³ is as hereinabove-defined in the presence of a chlorinated agent such as phosphorus oxychloride, phosphorus pentachloride or thionyl chloride, at a temperature from 60° C. to 120° C.

The 6-aminopyrimidin-4-ol compounds of formula (VI) are in turn obtained by reaction of the carboxyamidines of formula (II) with ethylcyanoacetate. The reaction is carried out in a solvent such as methanol, ethanol, isopropyl alcohol, butyl alcohol or tetrahydrofuran, in the presence of a base, such as sodium methoxide, sodium ethoxide or potassium tertbutoxide and at a temperature from room temperature to the boiling point of the solvent.

The resulting 6-aminopyrimidin-4-ol of formula (VI) can be reacted with a chlorinated agent such a phosphorus oxychloride, phosphorus pentachloride or a mixture of them, in a solvent such as phosphorus oxychloride, benzene or toluene, at a temperature from room temperature to the boiling point of the solvent to yield the 4-amino-6-chloropyrimidine compounds of formula (V). Optionally, the presence of a base such as dimethylaminoaniline, triethylamine or diisopropyl-ethylamine may be needed in this reaction step.

The compounds of formula (VII) can be obtained from the 6-chloropyrimidine-4-amines compounds of formula (V) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (VII) can also be prepared by reaction of amine (V) with an anhydride, at a temperature from 80° C. to 160° C.

Compounds of formula (I) and in particular those of formulas (XV) where R^{1′} and R¹″ are H, small alkyl or halogen and X is N or a carbon optionally substituted by a small alkyl, halogen and R² is an acyclic, monocyclic or polycyclic group linked to the pyrimidine ring through a nitrogen atom can be obtained as shown is Scheme 2.

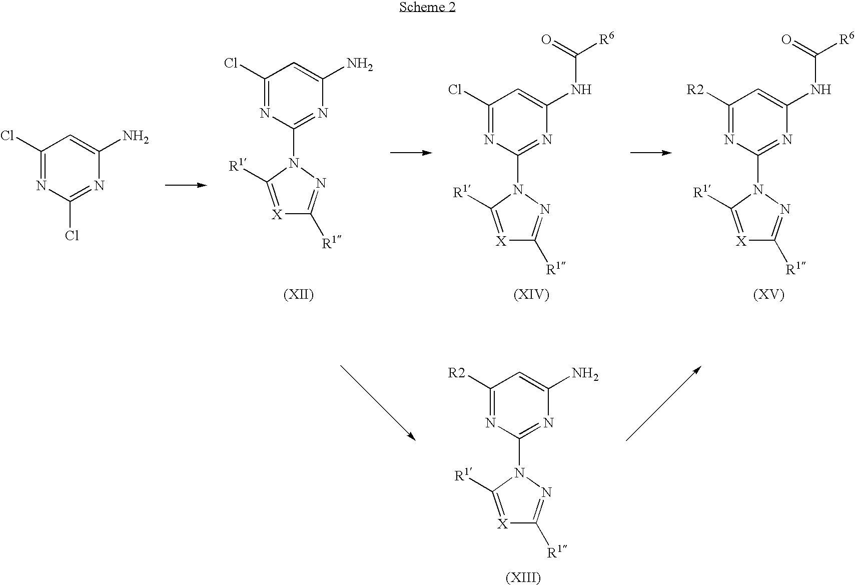

The compounds of formula (XII) can be obtained from 2,4-dichloro 6-aminopyrimidine by reaction with anhydrous hydrazine in the presence of a solvent such as NMP at a temperature of 60° C. then by reacting the intermediate with the appropriate diketone at a temperature from room temperature to 60° C.

The compounds of formula (XIV) can be obtained from the 6-chloropyrimidine-4-amines compounds of formula (XII) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (XIV) can also be prepared by reaction of amine (XII) with an anhydride, at a temperature from 80° C. to 160° C.

The resulting 6-chloropyrimidin-4-amine of formula (XIV) are reacted with a compound of formula R²—H wherein R² is a acyclic, monocyclic or polycyclic group linked to the pyrimidine ring through a nitrogen atom to yield the compounds of formula (XV) which is a particular case of the compounds of formula (I) according to the invention. The reaction is carried out in a solvent such as dioxane, dimethylformamide, dimethylacetamide or dimethylsulfoxide, in the presence of a base, such as sodium hydride, potassium carbonate or cesium carbonate, at a temperature from 60° C. to 140° C.

The 6-chloropyrimidin-4-amides of formula (XII) are reacted with a compound of formula R²—H wherein R² is an acyclic, monocyclic or polycyclic group linked to the pyrimidine ring through a nitrogen atom to yield the compounds of formula (XIII). The reaction is carried out in a solvent such as dioxane, dimethylformamide, dimethylacetamide or dimethylsulfoxide, in the presence of a base, such as sodium hydride, potassium carbonate or cesium carbonate, at a temperature from 60° C. to 140° C.

The compounds of formula (XV), which is a particular case of the compounds of formula (I) according to the invention, can be obtained from the 4-aminopyrimidine compounds of formula (XIII) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (XV) can also be prepared by reaction of amine (XVI) with an anhydride, at a temperature from 80° C. to 160° C.

Compounds of formula (I) and in particular those of formulas (XVIII) where R^{1′} and R^{1″} are H, small alkyl, halogen, X is a nitrogen or a carbon optionally substituted by H, small alkyl or halogen, R² is a acyclic, monocyclic or polycyclic group linked to the pyrimidine ring through a nitrogen atom and R³ is COR^{E}, OR⁶, COR⁶R⁷ or COOR⁶ can be obtained as shown is Scheme 3.

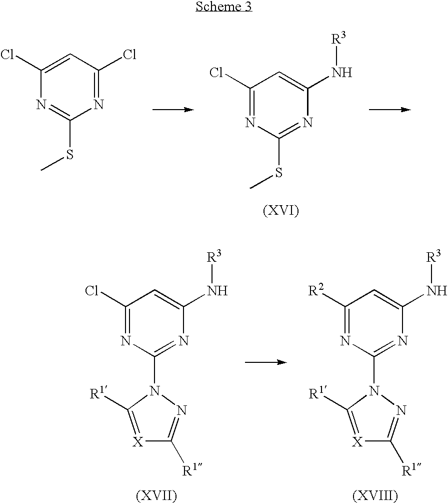

When R³ is an alkoxy, compounds of formula (XVI) can be obtained from the 4,6-dichloro-2-(methylthio)pyrimidine by reaction of a salt of an N-alkoxyamine and a base, such as pyridine, triethylamine or diisopropylethylamine, in a solvent such as tetrahydrofuran, dimethylformamide or dioxane, at a temperature from room temperature to the boiling point of the solvent. When R³ is COR^{E}, compounds of formula (XVI) can be prepared by reaction of the 4,6-dichloro-2-(methylthio)pyrimidine with ammonium hydroxide in a solvent such as methanol, ethanol, isopropyl alcohol or tetrahydrofuran, at a temperature from 80° C. to 140° C. followed by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (XVI) can also be prepared by reaction of the 4,6-dichloro 2-methylthiolpyrimidine with ammonium hydroxide in a solvent such as methanol, ethanol, isopropyl alcohol or tetrahydrofuran, at a temperature from 80° C. to 140° C. followed by reaction with an anhydride, at a temperature from 80° C. to 160° C.

The compounds of formula (XVII) can be obtained by oxidation of the methylthiol to the sulfone in presence of an oxidazing reagent such as OXONE®, hydrogen peroxide, potassium permanganate or sodium perborate. The sulfone intermediate is then reacted with anhydrous hydrazine in the presence of a solvent such as NMP at a temperature of 60° C. and reacted with the appropriate diketone at a temperature from room temperature to 60° C.

The resulting 6-chloropyrimidines of formula (XVII) are reacted with a compound of formula R²—H wherein R² is an acyclic, monocyclic or polycyclic group linked to the pyrimidine ring through a nitrogen atom to yield the compounds of formula (XVIII) which is a particular case of the compounds of formula (I) according to the invention. The reaction is carried out in a solvent such as dioxane, dimethylformamide, dimethylacetamide or dimethylsulfoxide, in the presence of a base, such as sodium hydride, potassium carbonate or cesium carbonate, at a temperature from 60° C. to 140° C.

Compounds of formula (I) and in particular those of formulas (XXI) where R^{1′} and R^{1″} are H, small alkyl or halogen, and R² is an acyclic, monocyclic or polycyclic group linked to the pyrimidine ring through a nitrogen atom can be obtained as shown is Scheme 4.

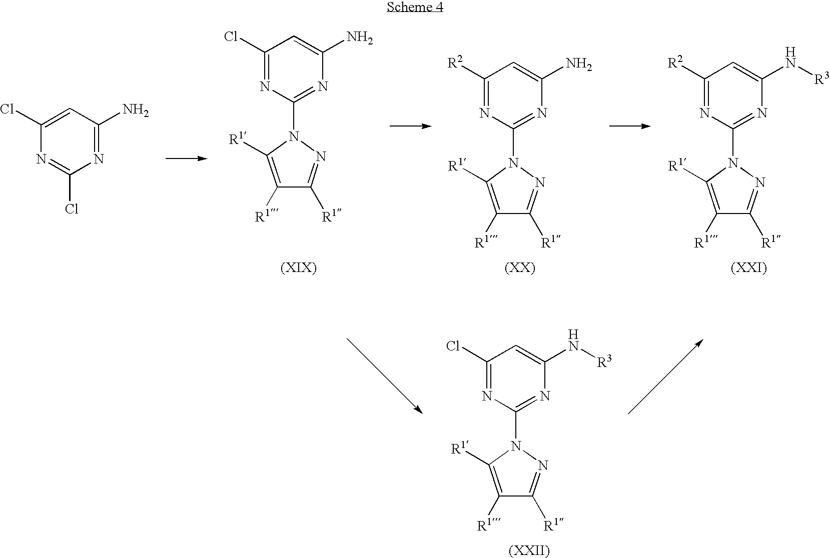

The compounds of formula (XIX) can be obtained by reacting the 4-amino-2,6-dichloropyrimidine with an optionally substituted pyrazole. The reaction is carried out in a solvent such as dioxane, dimethylformamide, dimethylacetamide or dimethylsulfoxide, in the presence of a base, such as sodium hydride, potassium carbonate or cesium carbonate, at a temperature from 60° C. to 140° C.

The resulting 6-chloropyrimidin-4-amines of formula (XIX) are reacted with a compound of formula R²—H wherein R² is an acyclic, monocyclic or polycyclic group linked to the pyrimidine ring through a nitrogen atom to yield the compounds of formula (XX). The reaction is carried out in a solvent such as dioxane, dimethylformamide, dimethylacetamide or dimethylsulfoxide, in the presence of a base, such as sodium hydride, potassium carbonate or cesium carbonate, at a temperature from 60° C. to 140° C.

The compounds of formula (XXI) can be obtained from the 2-pyrazolopyrimidine-4-amines compounds of formula (XX) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. When R³ is a heterocycle, general Buchwald conditions are used for the coupling. Compounds of formula (XXI), which is a particular case of the compounds of formula (I) according to the invention, can also be prepared by reaction of amine (XX) with an anhydride, at a temperature from 80° C. to 160° C.

The compounds of formula (XXII) can be obtained from the 6-chloropyrimidine-4-amines compounds of formula (XIX) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (XXII) can also be prepared by reaction of amine (XX) with an anhydride, at a temperature from 80° C. to 160° C.

The resulting 6-chloro2-pyrazolopyrimidines of formula (XXII) are reacted with a compound of formula R²—H wherein R² is an acyclic, monocyclic or polycyclic group linked to the pyrimidine ring through a nitrogen atom to yield the compounds of formula formula (XXI), which is a particular case of the compounds of formula (I) according to the invention. The reaction is carried out in a solvent such as dioxane, dimethylformamide, dimethylacetamide or dimethylsulfoxide, in the presence of a base, such as sodium hydride, potassium carbonate or cesium carbonate, at a temperature from 60° C. to 140° C.

The synthesis of amides of formulae (XXIII) and (XXIV) can be prepared following

Scheme 5.

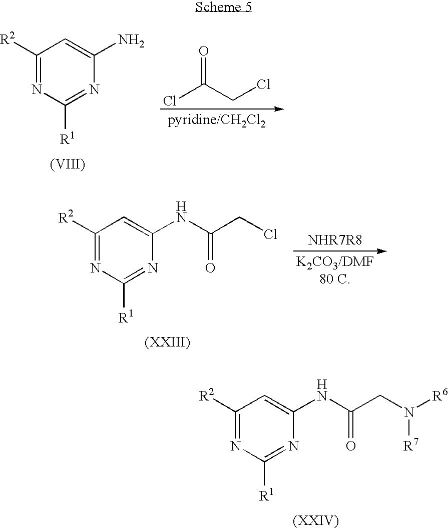

The amides of formula (XXIII) are obtained by reaction of a compound of formula (VIII) with chloroacetyl chloride in a solvent such as dichloromethane and base (e.g., pyridine). The resultant compound of formula (XXIII) is reacted with the desired amine (e.g., NHR⁶R⁷) in the presence of potassium carbonate and DMF to yield the desired amide of formula (XXIV). The same sequence of steps can be used starting from V, VI, XII, XIII, 4,6-dichloro-2-methylthiolpyrimidine, XIX or XX.

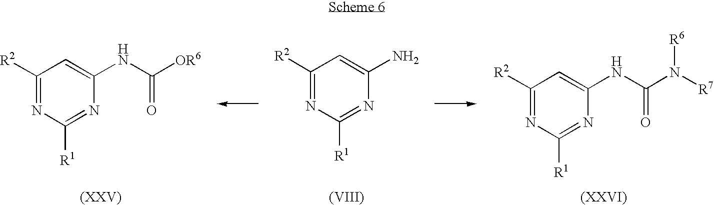

The carbamates of formula (XXV) are obtained by reaction of a compound of formula (VIII) with a compound of formula Z—COOR⁶, wherein Z represents a leaving group such as halogen atom, preferably chlorine or a group selected from ethoxy, methoxy, p-nitrophenoxy and imidazolyl. The reaction is carried out in a solvent, such as tetrahydrofuran, chloroform, methylene chloride or dimethylformamide, in the presence of a base, preferably triethylamine, diisopropylethylamine, potassium carbonate or sodium hydroxide, at a temperature from −70° C. to 100° C.

The carbamates of formula (XXV) are obtained by reaction of a compound of formula (VIII) with triphosgene, phosgene and an alcohol of formula HO—R⁶. The reaction is carried out in a solvent, such as tetrahydrofuran, chloroform, methylene chloride or dimethylformamide, in the presence of a base, preferably pyridine, at a temperature from −5° C. to 50° C.

The compounds of formula (VIII) can also be converted to the ureas of formula (XXVI) wherein R⁷ is a hydrogen atom by reaction with an isocyanate of formula R⁶—N═C═O in a solvent such as benzene, toluene or xylene, at a temperature from room temperature to 140° C.

The ureas of formula (XXVI) are obtained by reaction of a compound of formula (VIII) with triphosgene, phosgene and an amine of formula HNR⁶R⁷. The reaction is carried out in a solvent, such as tetrahydrofuran, chloroform, methylene chloride or dimethylformamide, in the presence of a base, preferably pyridine, at a temperature from −5° C. to 50° C.

The same sequence of steps can be used starting from V, VI, XII, XIII, 4,6-dichloro-2-methylthiolpyrimidine, XIX or XX.

When the defined groups R¹ to R⁷ are susceptible to chemical reaction under the conditions of the hereinbefore described processes or are incompatible with said processes, conventional protecting groups may be used in accordance with standard practice, for example see T. W. Greene and P. G. M. Wuts in ‘Protective Groups in Organic Chemistry’, 3^{rd} Edition, John Wiley & Sons (1999). It may be that deprotection will form the last step in the synthesis of compounds of formula (I).

### Pharmacological Activity

### Adenosine A₂ₐ Receptor Binding Assays

### Receptor Cloning

The coding sequence of the human A_{2A} receptor was amplified from a human brain cDNA library by the polymerase chain reaction. The amplicon was cloned into the pcDNA5/FRT/V5-His-TOPO expression vector (Invitrogen) and sequence confirmed using an ABI 3100 automated sequencer (Applied Biosystems). The expression construct was transfected into Flp-In HEK cells (Invitrogen) using Lipofectamine 2000 (Invitrogen). Cells stably expressing the human A_{2A} receptor were selected using 1 mg/ml hygromycin in complete DMEM.

### Membrane Preparation

Crude membranes were prepared from Flp-In HEK cells transfected with the human A_{2A} receptor by resuspending cells in lysis buffer (50 mM Tris-HCl pH 7.4, 5 mM EDTA, 10 mM MgCl₂) and disrupting under N₂ at a pressure of 900 psi (Parr Cell disruption bomb, cat.4639) for 30 min on ice followed by differential centrifugation. The resulting crude membrane pellet was resuspended in assay buffer (50 mM Tris HCl pH 7.4, 1 mM EDTA, 10 mM MgCl₂). Membrane protein concentration was determined by Bradford assay and aliquots were stored at −80° C.

### Binding Assay

An aliquot of membranes (5-10 μg of protein) was pre-incubated for 30 min at RT in the presence of 10 μg/ml Adenosine Deaminase (Type IV Calf Spleen, Sigma). Membranes were then incubated for 90 min with 1.0 nM [³H]-ZM 241385 (27.40 Ci/mmol Tocris R1036) in the presence of varying concentrations of competing ligand. Non-specific binding was determined in the presence of excess (1 μM) of CGS15943. Bound and free ligand were separated by rapid vacuum filtration using a Packard 96-well cell harvester onto UniFilter GF/C filter plates (PerkinElmer) that had been pretreated with 0.5% polyethyleneimine. The filter plates were than washed 3×200 μl with 50 mM Tris HCl, 50 mM NaCl pH 7.4. Bound radioligand was determined by scintillation counting using a TopCount-NXT (Packard). Binding data was analyzed by nonlinear, least-squares curve fitting algorithms using GraphPad Prism (GraphPad Software, Inc. San Diego, Calif.) or ActivityBase (IDBS, Guildford, Surrey, UK). Kᵢ values were calculated from IC₅₀ values using the Cheng-Prusoff equation (Cheng, Y, Prusoff, W. H. Biochem. Pharm. 22:3099-3108, 1973.).

For A_{2A} membrane assay:
ZM241385 measured Kd=0.3±0.2 nM; Bₘₐₓ=33±8 μmol/mg by Scatchard Analysis

### Binding Ki=0.25±0.04 nM.

With reference to A_{2A} receptor binding affinities, A_{2A} receptor antagonists of this invention may have a Ki of less than 10 μM. In one embodiment of this invention, an A_{2A} receptor antagonist has a Ki of less than 1 μM. In another embodiment an A_{2A} receptor antagonist has a Ki of less than 100 nM, and in still another embodiment an A_{2A} receptor antagonist has a Ki of less than 10 nM.

The pyrimidin-4-amine derivatives of the invention are useful in the treatment or prevention of diseases known to be susceptible to improvement by treatment with an antagonist of an adenosine receptor, in particular those susceptible to improvement by treatment with and antagonist of the A_{2A} adenosine receptor. Such diseases are, for example ischemia, supraventricular arrhythmias, acute renal failure, myocardial reperfusion injury, allergic reactions including but not limited to rhinitis, urticaria, scleroderm arthritis, other autoimmune diseases, inflammatory bowel diseases, asthma, diabetes mellitus, obesity, Parkinson disease, Huntington's disease, dystonias such as restless leg syndrome, dyskinesias such as those caused by prolonged use of neuroleptic and dopaminergic drugs or sleep disorders.

Accordingly, compounds of the invention and pharmaceutically acceptable salts thereof, and pharmaceutical compositions comprising such compound and/or salts thereof, may be used in a method of treatment of disorders of the human body which comprises administering to a subject requiring such treatment an effective amount of a compound of the invention or a pharmaceutically acceptable salt thereof.

The present invention also provides pharmaceutical compositions which comprise, as an active ingredient, a compound of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight of the composition depending upon the nature of the formulation and whether further dilution is to be made prior to application. Preferably the compositions are made up in a form suitable for oral, topical, nasal, rectal, percutaneous or injectable administration.

The pharmaceutically acceptable excipients which are admixed with the active compound, or salts of such compound, to form the compositions of this invention are well-known per se and the actual excipients used depend inter alia on the intended method of administering the compositions.

Compositions of this invention are preferably adapted for injectable and per os administration. In this case, the compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Effective doses are normally in the range of 2-2000 mg of active ingredient per day. Daily dosage may be administered in one or more treatments, preferably from 1 to 4 treatments, per day.

The present invention will be further illustrated by the following examples. The examples are given by way of illustration only and are not to be construed as a limiting.

Reagents, starting materials, and solvents were purchased from commercial suppliers and used as received. Concentration refers to evaporation under vacuum using a Büchi rotatory evaporator. Reaction products were purified, when necessary, by flash chromatography on silica gel (40-63 μm) with the solvent system indicated. Spectroscopic data were recorded on a Varian Mercury 300 MHz Spectrometer and a Bruker Avance 500 MHz spectrometer.

### Analytical HPLC-MS Method 1

Platform: Agilent 1100 series: equipped with an auto-sampler, an UV detector (220 nM and 254 nM), a MS detector (APCI);
HPLC column: Phenomenex Synergi-Max RP, 2.0×50 mm column;

HPLC gradient: 1.0 mL/minute, from 5% acetonitrile in water to 95% acetonitrile in water in 13.5 minutes, maintaining 95% for 2 minute. Both acetonitrile and water have 0.025% TFA.

### Analytical HPLC-MS Method 2

Platform: Dionex: equipped with an autosampler, an UV detector (220 nM and 254 nM), a MS detector (APCI);
HPLC column: Phenomenex CX18 4.6×150 mm;
HPLC gradient: 95% 0.04% NH₄OH/H₂O to 90% 0.04% NH₄OH/ACN over 9.86 min, 12.30 min run

### Analytical HPLC-MS Method 3

Platform: Agilent 1100 series: equipped with an auto-sampler, an UV detector (220 nM and 254 nM), a MS detector (APCI);
HPLC column: Phenomenex Synergi-Max RP, 2.0×50 mm column;
HPLC gradient: 1.0 mL/minute, from 10% acetonitrile in water to 90% acetonitrile in water in 2.5 minutes, maintaining 90% for 1 minute. Both acetonitrile and water have 0.025% TFA.

### Analytical HPLC-MS Method 4

Platform: Agilent 1100 series: equipped with an auto-sampler, an UV detector (230 nM and 254 nM), a MS detector (APCI);
HPLC column: Phenomenex Synergi-Max RP, 2.0×50 mm column;
HPLC gradient: Solvent C is 6 mM Ammonium Formate in water, solvent D is 25% Acetonitrile in Methanol. The gradient runs from 5% D (95% C) to 95% D (5% C) in 6.43 min with a 1.02 min hold at 95% D followed by a return and hold at 5% D for 1.52 min.

### Intermediate 1: 6-Chloro-2-(3,5-dimethyl-pyrazol-1-yl)-pyrimidin-4-ylamine

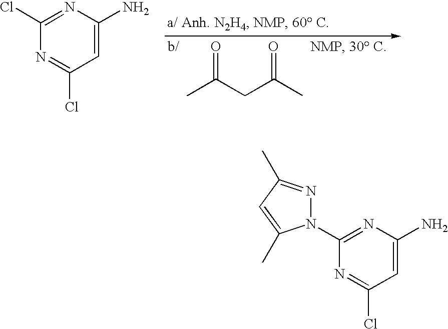

40.0 g (0.24 mol, 1 eq) of 4-amino-2,6-dichloropyrimidine was dissolved in 200 mL of N-methylpyrrolidinone. The slurry was heated to 60° C. and 19.14 mL (0.61 mol, 2.5 eq.) of anhydrous hydrazine was added slowly. After 1.5 hours, the addition was complete. The reaction was cooled down to room temperature and 62.6 mL (0.61 mol, 2.5 eq.) of 2,4-pentanedione was added slowly, keeping the reaction temperature below 50° C. After one hour, the reaction was heated again at 50° C. then 200 mL of ethyl alcohol were added, followed by 400 mL of water. Once the water addition was complete, the reaction mixture was cooled to room temperature, filtered on paper. The cake was washed with alcohol/water (3×200 mL) and dried under vacuum at 60° C. overnight. The recovered tan solid was a mixture of the desired regioisomer (43.2 g, 85 area % purity at 254 nm) and the 4-dimethylpyrazole regioisomer. The product was recrystallized from hot THF/i-PrOAc to give a white solid (yield 66%). LCMS (Method 3) m/z 223.9 [MH+], Tr=1.97 min

### Intermediate 2. 6-Chloro-2-(3,4,5-trimethyl-pyrazol-1-yl)-pyrimidin-4-ylamine

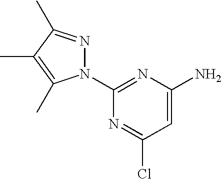

Intermediate 2 was produced using the same procedure as described above for Intermediate 1 using 3-methyl-2,4-pentanedione instead of 2,4-pentanedione. LCMS (Method 3) m/z 237.9 [MH+], Tr=2.38 min

### Intermediate 3. 6-Chloro-2-(4-chloro-3,5-dimethyl-pyrazol-1-yl)-pyrimidin-4-ylamine

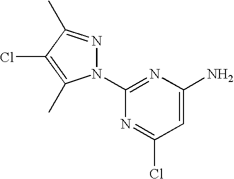

Intermediate 3 was produced using the same procedure as described above for Intermediate 1 using 3-chloro-2,4-pentanedione instead of 2,4-pentanedione. LCMS (Method 3) m/z 257.7 [MH+], Tr=2.61 min

### Intermediate 4. 6-Chloro-2-(3,5-dimethyl-[1,2,4]triazol-1-yl)-pyrimidin-4-ylamine

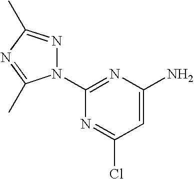

Intermediate 4 was produced using the same procedure as described above for Intermediate 1 using diacetamide instead of 2,4-pentanedione. LCMS (Method 3) m/z 224.8 [MH+], Tr=1.96 min

### Intermediate 5: N-[6-Chloro-2-(3,5-dimethyl-pyrazol-1-yl)-pyrimidin-4-yl]-acetamide

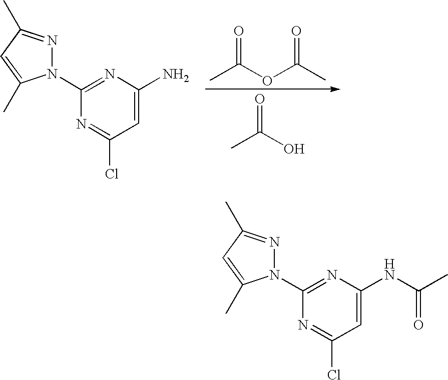

40.0 g (0.18 mol, 1 eq.) of 6-Chloro-2-(3,5-dimethyl-pyrazol-1-yl)-pyrimidin-4-ylamine was dissolved in 200 mL (0.9 mol, 5 eq.) acetic acid and stirred at r.t. 80 mL (0.8 mol, 4.7 eq.) of acetic anhydride was added and the mixture was heated at 90° C. overnight. Once the reaction was complete, it was cooled to room temperature and 16 mL of water was added over 30 minutes. The mixture was then filtered through filter paper and the cake was washed with water (4×75 mL). The solid was dried in a vacuum oven at 50° C. overnight. The AcOH solvate (48.2 g, 0.15 mol, 83% yield) was an off-white crystalline solid. LCMS (Method 3) m/z 265.9 [MH+], Tr=2.11 min

### Intermediate 6. N-[6-Chloro-2-(3,4,5-trimethyl-pyrazol-1-yl)-pyrimidin-4-yl]-acetamide

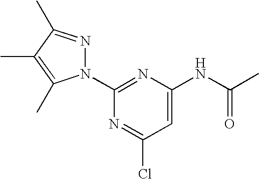

Intermediate 6 was prepared according to the procedures described in Intermediate 5 using Intermediate 2 instead of 6-Chloro-2-(3,5-dimethyl-pyrazol-1-yl)-pyrimidin-4-ylamine. LCMS (Method 3) m/z 279.8 [MH+], Tr=2.55 min

### Intermediate 7. N-[6-Chloro-2-(4-chloro-3,5-dimethyl-pyrazol-1-yl)-pyrimidin-4-yl]-acetamide

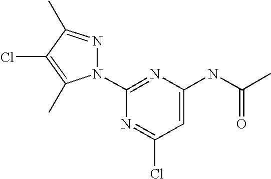

Intermediate 7 was prepared according to the procedures described in Intermediate 5 using Intermediate 3 instead of 6-Chloro-2-(3,5-dimethyl-pyrazol-1-yl)-pyrimidin-4-ylamine. LCMS (Method 3) m/z 299.8 [MH+], Tr=2.71 min

### Intermediate 8. N-[6-Chloro-2-(3,5-dimethyl-[1,2,4]triazol-1-yl)-pyrimidin-4-yl]-acetamide

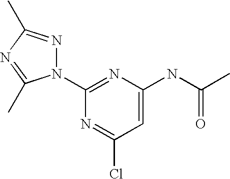

Intermediate 7 was prepared according to the procedures described in Intermediate 5 using Intermediate 4 instead of 6-Chloro-2-(3,5-dimethyl-pyrazol-1-yl)-pyrimidin-4-ylamine. LCMS (Method 3) m/z 266.8 [MH+], Tr=2.18 min

### Intermediate 9: N-[6-Chloro-2-(3,5-dimethyl-pyrazol-1-yl)-pyrimidin-4-yl]-propionamide

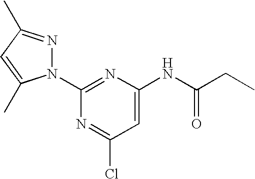

1.4 mL (16.3 mmol, 1.2 eq.) of propionyl chloride were added slowly to a cold DMF solution (30 mL) of 1.3 mL (16.3 mmol, 1.2 eq.) of anhydrous pyridine and 3.0 g (13.6 mmol, 1 eq.) of Intermediate 1. The mixture was stirred at room temperature overnight. After completion of the reaction, the reaction mixture was neutralized to pH 7 with a saturated aqueous solution of sodium bicarbonate and the product extracted with dichloromethane (3×50 mL). The organic layers were combined, dried over magnesium sulfate and concentrated. Purification by column chromatography with silica gel and methylene chloride with 2% methanol as eluent gave 3.4 g (75% yield) of a light brown solid. LCMS (Method 1) m/z 280.0 [MH+], Tr=6.08 min

### Intermediate 10: N-[6-Chloro-2-(3,5-dimethyl-pyrazol-1-yl)-pyrimidin-4-yl]-3-methyl-butyramide

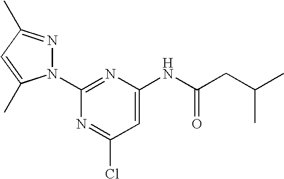

Intermediate 1 was prepared by reacting Intermediate 1 with isobutyryl chloride in a similar way as for intermediate 9. The residue was purified by liquid chromatography using a mixture of 1 to 1 ethyl acetate/hexanes to afford a white solid in similar yields. LCMS (Method 1) m/z 308.1 [MH+], Tr=7.44 min

### Intermediate 11: 2-Chloro-N-[6-chloro-2-(3,5-dimethyl-pyrazol-1-yl)-pyrimidin-4-yl]-acetamide

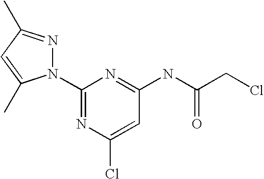

To a solution of Intermediate 1 (1.9 g, 8.7 mmol, 1 eq.) in dichloromethane (100 mL) was added pyridine (0.9 mL, 11.7 mmol, 1.3 eq.) followed by a dropwise addition of chloroacetyl chloride (1.1 mL, 13.5 mmol, 1.5 eq.) at 0° C. The mixture was allowed to warm up to room temperature overnight with stirring. After the completion of the reaction, the solution was cooled to 0° C. with a ice bath and carefully quenched with 25 mL of an aqueous saturated solution of NaHCO₃. The reaction was extracted with dichloromethane (3×25 mL). The combined organic layer was washed with brine, dried with magnesium sulfate, filtered, and concentrated. The residue was purified by silica gel chromatography using dichloromethane with 10% methanol and afforded 2-Chloro-N-[4-chloro-2-(3,5-dimethyl-pyrazol-1-yl)-pyrimidin-4-yl]-acetamide in 92% yield as a yellow solid. LCMS (Method 3) m/z 300.0 [MH+], Tr=2.69 min.

### Intermediate 12: N-[6-Chloro-2-(3,5-dimethyl-pyrazol-1-yl)-pyrimidin-4-yl]-2-morpholin-4-yl-acetamide

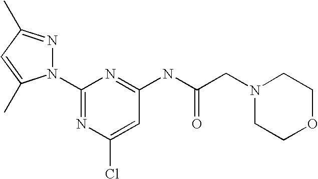

Intermediate 11 (3.0 g, 13 mmol, 1 eq.) was dissolved in dichloromethane (25 mL). Morpholine (1.2 mL, 14 mmol, 1.1 eq.) and diisopropylamine (4.6 mL, 26 mmol, 2.0 eq.) were added dropwise at room temperature. After stirring overnight, the solution was partitioned with water and extracted with dichloromethane (3×25 mL). The combined organic layers were washed with brine (50 mL), dried, filtered and concentrated under reduced pressure. The residue was purified by silica gel chromatography using methylene chloride as eluent with a gradient of methanol (2 to 5%) to afford 1.5 g (33% yield) of Intermediate 12. LCMS (Method 3) m/z 300.0 [MH+], Tr=2.69 min.

### Intermediate 13: N-[6-Chloro-2-(3,5-dimethyl-pyrazol-1-yl)-pyrimidin-4-yl]-2-(4-methyl-piperazin-1-yl)-acetamide

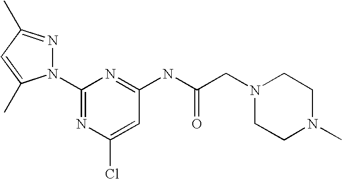

Intermediate 13 was prepared by reacting Intermediate 11 with N-methyl piperidine in a similar way as for Intermediate 12. LCMS (Method 2) m/z 363.6 [MH+], Tr=7.34 min

### Intermediate 14: N-[6-Chloro-2-(3,5-dimethyl-pyrazol-1-yl)-pyrimidin-4-yl]-2-(pyrrolidin-1-yl)-acetamide

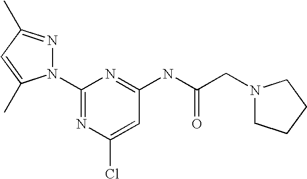

Intermediate 14 was prepared by reacting Intermediate 11 with pyrrolidine in a similar way as for Intermediate 12.

### Compound 1-3: N-[2-(3,5-Dimethyl-pyrazol-1-yl)-6-(2-(R)-methoxymethyl)pyrrolidin-1-yl-pyrimidin-4-yl]-acetamide

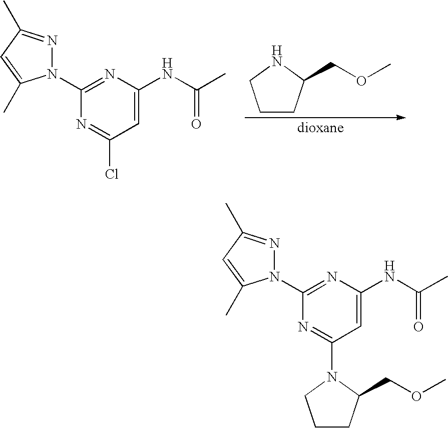

Intermediate 5 (50 mg, 0.19 mmol, 1 eq.) was dissolved in dry dioxane (2 mL). 1.2 eq (26 mg, 0.23 mmol) of (R)-2-methoxymethyl pyrrolidine was added. The mixture was heated at 80° C. for 2 hours, cooled down to room temperature, filtered and purified by HPLC. LCMS (Method 2) m/z 345.0 [MH+], Tr=6.23 min

### Compound 1-14: N-[2-(3,5-Dimethyl-pyrazol-1-yl)-6-(2-oxo-pyrrolidin-1-yl)-pyrimidin-4-yl]-acetamide

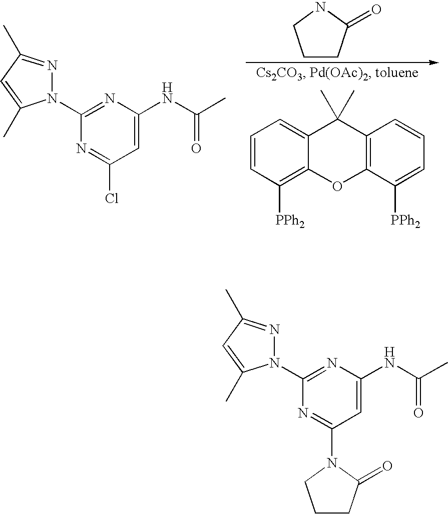

A mixture of Intermediate 4 (50 mg, 0.19 mmol, 1 eq.), lactame (81 mg, 0.95 mmol, 5 eq.), palladium acetate (5 mg, 0.02 mmol, 0.1 eq), 4,5-bis(diphenylphosphino)-9,9-dimethylxanthene (17 mg, 0.03 mmol, 0.15 eq), cesium carbonate (68 mg, 0.21 mmol, 1.1 eq) was heated in dry toluene (2 mL) at 100° C. overnight. After return to room temperature and filtration, the mixture was purified by HPLC. LCMS (Method 2) m/z 315.2 [MH+], Tr=5.31 min

### Compound 1-51: N-[2-(3,5-Dimethyl-pyrazol-1-yl)-6-pyrrolidin-1-yl-pyrimidin-4-yl]-acetamide

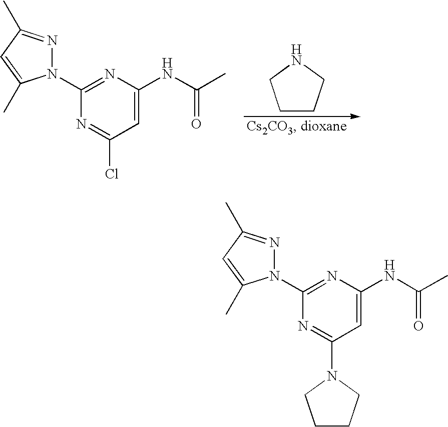

Intermediate 5 (50 mg, 0.19 mmol, 1 eq.) was dissolved in dry dioxane (2 mL). 1.1 eq. (68 mg, 0.21 mmol) of cesium carbonate and 1.1 eq (15 mg, 0.21 mmol) of pyrrolidine were added. The mixture was heated at 80° C. until completion, cooled down to room temperature, filtered and purified by HPLC. LCMS (Method 1) m/z 301.1 [MH+], Tr=4.88 min

The compounds of Table 1A were prepared by reacting the appropriate intermediate described above with the appropriate amine representing the R² substituent:

TABLE 1A

| | | | | | Reten | |
|---|---|---|---|---|---|---|
| Cmpd | | | | MS | time | HPLC |
| No. | InterMediate | R² | MW | ION | (min) | Method |
| 1-1 | 5 | 3-fluoro-2-methoxy- | 370.4 | 371.3 | 8.1 | 2 |
| | | phenylamino | | | | |
| 1-2 | 5 | 3,4-Dihydro-1H- | 362.4 | 363.3 | 10.1 | 2 |
| | | isoquinolin-2-yl | | | | |
| 1-3 | 5 | (R)-2-methoxymethyl- | 362.4 | 362.9 | 7.6 | 1 |
| | | pyrrolidin-1-yl | | | | |
| 1-4 | 5 | 2-methyl-2,3-dihydro-indol-1- | 344.4 | 345.2 | 6.4 | 2 |
| | | yl | | | | |
| 1-5 | 5 | 2-oxo-2,3-dihydro-indol-1-yl | 358.4 | 359.1 | 8.0 | 2 |
| 1-6 | 5 | 3-methoxy-phenylamino | 362.4 | 363.1 | 6.2 | 1 |
| 1-7 | 5 | Phenylamino | 352.4 | 352.8 | 7.8 | 2 |
| 1-8 | 5 | 1,3-Dihydro-isoindol-2-yl | 322.4 | 323.8 | 7.7 | 2 |
| 1-9 | 5 | 2-methoxymethyl-pyrrolidin- | 401.5 | 402.3 | 8.7 | 2 |
| | | 1-yl | | | | |
| 1-10 | 5 | 2-oxo-piperidin-1-yl | 344.4 | 344.8 | 5.0 | 1 |
| 1-11 | 5 | 2-fluoro-phenylamino | 328.4 | 329.6 | 5.0 | 1 |
| 1-12 | 5 | 2-methoxy-phenylamino | 340.4 | 340.8 | 7.6 | 2 |
| 1-13 | 5 | (2R,5R)-2,5-Bis- | 352.4 | 352.8 | 7.7 | 2 |
| | | methoxymethyl-pyrrolidin-1- | | | | |
| | | yl | | | | |
| 1-14 | 5 | 2-oxo-pyrrolidin-1-yl | 388.5 | 389.1 | 7.5 | 1 |
| 1-15 | 5 | 2-propyl-pyrrolidin-1-yl | 429.5 | 429.9 | 4.4 | 1 |
| 1-16 | 5 | Bis-(2-methoxy-ethyl)-amino | | | | 1 |
| 1-17 | 5 | (2R,4R)-4-methoxy-2- | 412.4 | 412.8 | 6.3 | 1 |
| | | methoxymethyl-pyrrolidin-1- | | | | |
| | | yl | | | | |
| 1-18 | 5 | (3-Hydroxy-3-phenyl-propyl)- | | | | 1 |
| | | methyl-amino | | | | |
| 1-19 | 5 | 3,6-Dihydro-2H-pyridin-1-yl | 374.4 | 375.1 | 4.8 | 1 |
| 1-20 | 5 | 3-methoxymethyl-piperidin-1- | 312.4 | 312.8 | 7.7 | 1 |
| | | yl | | | | |
| 1-21 | 5 | (R)-2-hydroxymethyl- | 358.4 | 358.7 | 7.7 | 1 |
| | | pyrrolidin-1-yl | | | | |
| 1-22 | 5 | 2-propyl-pyrrolidin-1-yl | | | | 1 |
| 1-23 | 5 | 3,5-Dimethyl-piperidin-1-yl | 330.4 | 330.8 | 4.2 | 1 |
| 1-24 | 5 | 3-oxo-2-aza-spiro[4.5]dec-2-yl | 342.4 | 343.3 | 7.8 | 2 |
| 1-25 | 5 | (R)-2-methoxymethyl- | 382.5 | 383.1 | 8.7 | 1 |
| | | piperidin-1-yl | | | | |
| 1-26 | 5 | 2-methyl-pyrrolidin-1-yl)- | 358.4 | 359.1 | 5.3 | 1 |
| | | pyrimidin-4-yl | | | | |
| 1-27 | 5 | (3-hydroxy-3-phenyl-propyl)- | 366.4 | 367.1 | 6.3 | 1 |
| | | methyl-amino | | | | |
| 1-28 | 5 | 4-methoxy-phenylamino | 455.0 | 455.0 | 5.4 | 1 |
| 1-29 | 5 | Bis-(2-methoxy-ethyl)-amino | 330.4 | 330.9 | 4.1 | 1 |
| 1-30 | 5 | 2-oxo-oxazolidin-3-yl | 362.4 | 363.0 | 8.1 | 2 |
| 1-31 | 5 | 3-methyl-piperidin-1-yl | 316.3 | 317.5 | 4.5 | 1 |
| 1-32 | 5 | 2-methoxymethyl-piperidin-1- | 328.4 | 329.0 | 7.4 | 2 |
| | | yl | | | | |
| 1-33 | 5 | isopropyl-(2-methoxy-ethyl)- | 386.5 | 387.4 | 9.1 | 2 |
| | | amino | | | | |
| 1-34 | 5 | Methoxyamino | 330.4 | 331.1 | 6.4 | 1 |
| 1-35 | 5 | 6-methoxy-pyridin-3-ylamino | 346.4 | 347.4 | 7.7 | 1 |
| 1-36 | 5 | ethyl-(2-methoxy-ethyl)- | 353.4 | 354.1 | 6.9 | 2 |
| | | amino | | | | |
| 1-37 | 5 | Ethoxyamino | 332.4 | 333.3 | 8.6 | 2 |
| 1-38 | 5 | 3-trifluoromethyl-5,6-dihydro- | 290.3 | 291.2 | 6.3 | 1 |
| | | 8H-[1,2,4]triazolo[4,3- | | | | |
| | | a]pyrazin-7-yl | | | | |
| 1-39 | 5 | (tetrahydro-furan-2-ylmethyl)- | 421.4 | 422.1 | 4.9 | 1 |
| | | amino | | | | |
| 1-40 | 5 | 2-ethyl-piperidin-1-yl | 413.5 | 414.2 | 4.5 | 1 |
| 1-41 | 5 | piperidin-1-yl | 342.4 | 343.3 | 7.7 | 2 |
| 1-42 | 5 | 3-methoxy-piperidin-1-yl | 314.4 | 314.9 | 5.4 | 1 |
| 1-43 | 5 | 2-methoxy-1-phenyl- | 344.4 | 344.9 | 4.8 | 1 |
| | | ethylamino | | | | |
| 1-44 | 5 | (2-hydroxy-ethyl)-methyl- | 380.4 | 381.4 | 7.6 | 1 |
| | | amino | | | | |
| 1-45 | 5 | [1,3]dioxolan-2-ylmethyl- | 304.4 | 304.9 | 5.4 | 2 |
| | | methyl-amino | | | | |
| 1-46 | 5 | Diethylamino | 346.4 | 346.9 | 7.9 | 2 |
| 1-47 | 5 | 2-carboxy-4-hydroxy- | 302.4 | 302.8 | 9.1 | 2 |
| | | pyrrolidin-1-yl | | | | |
| 1-48 | 5 | (R)-2-isopropoxymethyl- | 360.4 | 360.1 | 6.0 | 1 |
| | | pyrrolidin-1-yl | | | | |
| 1-49 | 5 | (2R,4R)-2-hydroxymethyl-4- | 372.5 | 372.9 | 5.1 | 1 |
| | | methoxy-pyrrolidin-1-yl | | | | |
| 1-50 | 5 | (S)-3-fluoro-pyrrolidin-1-yl | 360.4 | 361.0 | 4.0 | 1 |
| 1-51 | 5 | pyrrolidin-1-yl | 318.4 | 319.0 | 4.8 | 1 |
| 1-52 | 5 | 4-pyridin-2-yl-piperazin-1-yl | 372.5 | 373.0 | 7.8 | 2 |
| 1-53 | 5 | (R)-1-(tetrahydro-furan-2- | 332.5 | 333.2 | 9.0 | 2 |
| | | yl)methyl]-amino | | | | |
| 1-54 | 5 | 4,4-Diethyl-2-oxo-pyrrolidin- | 330.4 | 331.0 | 4.5 | 1 |
| | | 1-yl | | | | |
| 1-55 | 5 | 3-hydroxy-piperidin-1-yl | 372.5 | 373.5 | 7.8 | 1 |
| 1-56 | 5 | (R)-2-carboxylic acid methyl | 330.4 | 331.0 | 5.8 | 2 |
| | | ester-pyrrolidine-1-yl | | | | |
| 1-57 | 5 | (R)-3-Dimethylamino- | 358.4 | 358.8 | 6.9 | 1 |
| | | pyrrolidin-1-yl | | | | |
| 1-58 | 5 | Dimethylamino | 371.4 | 371.8 | 5.9 | 2 |
| 1-59 | 5 | (R)-3-Dimethylamino- | 358.4 | 358.9 | 4.7 | 1 |
| | | pyrrolidin-1-yl | | | | |
| 1-60 | 5 | 2-ethoxymethyl-pyrrolidin-1- | 330.4 | 331.0 | 4.5 | 1 |
| | | yl | | | | |
| 1-61 | 5 | 4-methoxymethyl-piperidin-1- | 358.4 | 358.9 | 5.5 | 1 |
| | | yl | | | | |
| 1-62 | 5 | (R)-2-(2-methoxy-ethyl)- | 358.4 | 358.7 | 7.5 | 2 |
| | | piperidin-1-yl | | | | |
| 1-63 | 5 | morpholin-4-yl | 405.5 | 406.5 | 8.5 | 2 |
| 1-64 | 5 | (S)-2-methoxymethyl- | 316.4 | 317.5 | 6.2 | 2 |
| | | pyrrolidin-1-yl | | | | |
| 1-65 | 5 | 4-phenyl-3,6-dihydro-2H- | 344.4 | 345.6 | 6.3 | 2 |
| | | pyridin-1-yl | | | | |
| 1-66 | 5 | 2,6-Dimethyl-morpholin-4-yl | 388.5 | 389.5 | 10.3 | 2 |
| 1-67 | 5 | (pyridin-4-ylmethyl)-amino | 342.5 | 342.9 | 9.2 | 2 |
| 1-68 | 5 | Azetidin-1-yl | 412.5 | 413.1 | 4.0 | 1 |
| 1-69 | 5 | ethyl-pyridin-4-ylmethyl- | 286.3 | 286.9 | 6.7 | 2 |
| | | amino | | | | |
| 1-70 | 5 | 2-ethoxymethyl-pyrrolidin-1- | 365.4 | 366.4 | 6.6 | 2 |
| | | yl | | | | |
| 1-71 | 5 | 4-phenyl-piperidin-1-yl | 358.4 | 359.4 | 8.0 | 2 |
| 1-72 | 5 | 4-methoxy-piperidin-1-yl | 328.4 | 328.9 | 8.3 | 1 |
| 1-73 | 5 | 2-carboxylic acid ethyl ester- | 344.4 | 345.2 | 6.8 | 2 |
| | | piperidine-1yl | | | | |
| 1-74 | 5 | (R)-2-(1-hydroxy-1-methyl- | 330.4 | 331.5 | 6.8 | 2 |
| | | ethyl)-pyrrolidin-1-yl | | | | |
| 1-75 | 5 | 1-oxo-1,3-dihydro- | 362.4 | 363.3 | 10.1 | 2 |
| | | isoindol-2-yl | | | | |
| 1-76 | 5 | 2-methoxymethyl-2,3- | 392.5 | 392.9 | 6.3 | 1 |
| | | dihydro-indol-1-yl | | | | |
| 1-77 | 9 | 2-methyl-2,3-dihydro- | 376.5 | 377.2 | 9.8 | 2 |
| | | indol-1-yl | | | | |
| 1-78 | 9 | (R)-2-methoxymethyl- | 358.4 | 359.1 | 8.0 | 2 |
| | | pyrrolidin-1-yl | | | | |
| 1-79 | 5 | (2S,4R)-4-methoxy-2- | 374.4 | 375.1 | 4.9 | 1 |
| | | methoxymethyl- | | | | |
| | | pyrrolidin-1-yl | | | | |
| 1-80 | 9 | 6-Cyano-3,4-dihydro-1H- | 401.5 | 402.3 | 8.7 | 2 |
| | | isoquinolin-2-yl | | | | |
| 1-81 | 12 | (R)-2-methoxymethyl- | 429.5 | 429.9 | 4.4 | 1 |
| | | pyrrolidin-1-yl | | | | |
| 1-82 | 1 | (R)-2-isopropoxymethyl- | 330.4 | 330.9 | 4.1 | 1 |
| | | pyrrolidin-1-yl | | | | |
| 1-83 | 5 | Acetyl-(3-fluoro-2-methoxy- | 412.4 | 412.8 | 6.3 | 1 |
| | | phenyl)-amino | | | | |
| 1-84 | 5 | (3-methoxy-phenyl)- | 366.4 | 367.1 | 6.3 | 1 |
| | | methyl-amino | | | | |
| 1-85 | 13 | (3-Chloro-phenylamino) | 455.0 | 455.0 | 5.4 | 1 |
| 1-86 | 5 | (S)-2-hydroxymethyl- | 330.4 | 330.9 | 4.1 | 1 |
| | | pyrrolidin-1-yl | | | | |
| 1-87 | 13 | (R)-2-methoxymethyl- | 442.6 | 443.3 | 7.5 | 2 |
| | | pyrrolidin-1-yl | | | | |
| 1-88 | 10 | (R)-2-methoxymethyl- | 386.5 | 387.4 | 9.1 | 2 |
| | | pyrrolidin-1-yl | | | | |
| 1-89 | 14 | (R)-2-methoxymethyl- | 413.5 | 414.2 | 4.5 | 1 |
| | | pyrrolidin-1-yl | | | | |
| 1-90 | 10 | (S)-2-hydroxymethyl- | 372.5 | 373.0 | 7.8 | 2 |
| | | pyrrolidin-1-yl | | | | |
| 1-91 | 5 | (1-phenyl-ethylamino)- | 350.4 | 351.5 | 8.1 | 2 |
| | | pyrimidin-4-yl | | | | |
| 1-92 | 5 | (S)-3-methoxy-pyrrolidin-1-yl | 330.4 | 331.0 | 4.5 | 1 |
| 1-93 | 10 | (R)-2-hydroxymethyl- | 372.5 | 373.4 | 7.8 | 1 |
| | | pyrrolidin-1-yl | | | | |
| 1-94 | 9 | (S)-3-Acetylamino-pyrrolidin- | 371.4 | 371.8 | 5.9 | 2 |
| | | 1-yl | | | | |
| 1-95 | 9 | morpholin-4-yl | 330.4 | 331.5 | 6.8 | 2 |
| 1-96 | 1 | (R)-2-methoxymethyl- | 302.4 | 302.9 | 7.0 | 2 |
| | | pyrrolidin-1-yl | | | | |
| 1-97 | 5 | (R)-3-methoxy-pyrrolidin-1-yl | 330.4 | 331.0 | 4.5 | 1 |
| 1-98 | 5 | 4-benzyl-piperazin-1-yl | 405.5 | 406.5 | 8.5 | 2 |
| 1-99 | 5 | 4-phenyl-piperazin-1-yl | 391.5 | 392.3 | 8.7 | 2 |
| 1-100 | 10 | pyrrolidin-1-yl | 342.4 | 342.9 | 9.2 | 2 |
| 1-101 | 13 | 2-oxo-pyrrolidin-1-yl | 412.5 | 413.1 | 4.0 | 1 |
| 1-102 | 12 | (S)-2-methoxymethyl- | 429.5 | 429.9 | 4.4 | 1 |
| | | pyrrolidin-1-yl | | | | |
| 1-103 | 1 | 4,4-diethyl-2-oxo- | 328.4 | 328.9 | 8.3 | 1 |
| | | pyrrolidin-1-yl | | | | |
| 1-104 | 10 | morpholin-4-yl | 358.4 | 359.2 | 7.9 | 2 |
| 1-105 | 5 | (RS)-2-methoxymethyl-4- | 358.4 | 359.0 | 5.6 | 4 |
| | | methyl-pyrrolidin-1-yl | | | | |
| 1-106 | 5 | (R)-2-methoxymethyl-4- | 359.4 | 357.0 | 5.6 | 4 |
| | | methylene-pyrrolidin-1-yl | | | | |
| 1-107 | 5 | (R)-2-methoxymethyl-4-oxo- | 358.4 | 358.9 | 4.5 | 4 |
| | | pyrrolidin-1-yl | | | | |
| 1-108 | 5 | (S)-4-hydroxy-(R)-2- | 360.4 | 361.0 | 4.3 | 4 |
| | | methoxymethyl- | | | | |
| | | pyrrolidin-1-yl | | | | |
| 1-109 | 5 | (R)-4-hydroxy-(R)-2- | 360.4 | 361.0 | 4.1 | 1 |
| | | methoxymethyl- | | | | |
| | | pyrrolidin-1-yl | | | | |
| 1-110 | 5 | (R)-4-benzyloxy-(R)-2- | 450.5 | 451.0 | 6.8 | 1 |
| | | methoxymethyl- | | | | |
| | | pyrrolidin-1-yl | | | | |
| 1-111 | 5 | (S)-2-benzyloxymethyl- | 420.5 | 421.0 | 6.2 | 1 |
| | | pyrrolidin-1-yl | | | | |
| 1-112 | 5 | (R)-2-benzyloxymethyl- | 420.5 | 421.0 | 6.2 | 1 |
| | | pyrrolidin-1-yl | | | | |
| 1-113 | 5 | (S)-2-(pyridin-2- | 407.5 | 408.0 | 5.7 | 1 |
| | | yloxymethyl)-pyrrolidine-1-yl | | | | |
| 1-114 | 5 | (R)-2-(pyridin-2- | 407.5 | 407.9 | 5.6 | 1 |
| | | yloxymethyl)-pyrrolidine-1-yl | | | | |
| 1-115 | 5 | 2-methoxymethyl-2-methyl- | 344.4 | 345.0 | 4.8 | 1 |
| | | pyrrolidin-1-yl | | | | |
| 1-116 | 1 | (S)-2-benzyloxymethyl- | 378.5 | 379.0 | 5.3 | 1 |
| | | pyrrolidin-1-yl | | | | |
| 1-117 | 5 | (R)-2-methylmethoxy-4- | 356.4 | 357.0 | 5.6 | 4 |
| | | methylene pyrrolidin-1-yl | | | | |

The compounds of Table 1B are made by reacting the appropriate intermediate described above with the appropriate amine representing the R³ substituent.

TABLE 1B

| Cmpd | Inter- | |
|---|---|---|
| No. | mediate | R³ |
| 1-200 | 5 | 2-methoxymethyl-3-methyl- |
| | | pyrrolidin-1-yl |
| 1-201 | 5 | 2-methoxymethyl-5-methyl- |
| | | pyrrolidin-1-yl |
| 1-202 | 5 | 2-methoxymethyl-4-ethoxy- |
| | | pyrrolidin-1-yl |
| 1-203 | 5 | 2-methoxymethyl-4-isopropyloxy- |
| | | pyrrolidin-1-yl |
| 1-204 | 5 | 4-(2-methoxyethoxyethoxy)- |
| | | 2-methoxymethyl-pyrrolidin- |
| | | 1-yl |
| 1-205 | 5 | (R)-2-methylmethoxy-4-[1- |
| | | phenyl-methylidene] |
| | | pyrrolidin-1-yl |

### Compound-1-96: 2-(3,5-Dimethyl-pyrazol-1-yl)-6-((R)-2-methoxymethyl-pyrrolidin-1-yl)-pyrimidin-4-ylamine

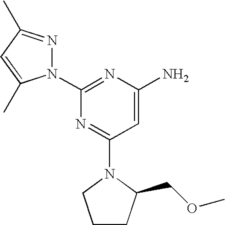

Intermediate 1 (25 mg, 0.11 mmol, 1 eq.) and (R)-2-methoxymethyl-pyrrolidine (23 mg, 0.27 mmol, 2.5 eq.) were heated in ethanol (1.5 mL) at 80° C. for 3 hours. After return to room temperature, the solution was filtered and purified by HPLC. LCMS (Method 2) m/z 302.9 [MH+], Tr=6.98 min

### Compound 1-118: [2-(3,5-Dimethyl-pyrazol-1-yl)-6-((R)-2-methoxymethyl-pyrrolidin-1-yl)-pyrimidin-4-yl]-carbamic acid ethyl ester

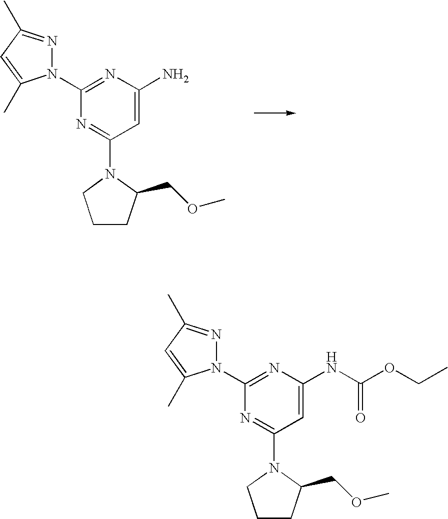

75 mg of triphosgene (0.25 mmol, 1 eq.) was added to 75 mg (0.25 mmol, 1 eq.) of Compound 1-96 and 0.02 mL (0.25 mmol, 1 eq.) of dry pyridine in 1 ml of dry dichloromethane at 0° C. After 30 minutes, dry ethanol was added and the solution was stirred at room temperature overnight. After completion of the reaction, solvents were evaporated, the residue dissolved in 1 mL methanol and purified by HPLC. LCMS (Method 1) m/z 375.1 [MH+], Tr=5.93 min

The compounds of Table 1C were prepared by reacting compound 1-96 with the appropriate amine or alcohol.

TABLE 1C

| | | | | | | |
|---|---|---|---|---|---|---|
| | | | | | Reten | |
| Cmpd | | | | MS | time | HPLC |
| No. | Reactant | R³ | MW | ION | (min) | Method |

| | | | | | | |
|---|---|---|---|---|---|---|
| 1-118 | 1-96 | ethylcarbamate | 374.4 | 375.1 | 5.9 | 1 |
| 1-119 | 1-96 | isopropylurea | 387.5 | 388.1 | 6.1 | 1 |
| 1-120 | 1-96 | Dimethylurea | 373.5 | 374.2 | 4.9 | 1 |

### Compound 1-121: [2-(3,5-Dimethyl-pyrazol-1-yl)-6-((R)-2-methoxymethyl-pyrrolidin-1-yl)-pyrimidin-4-yl]-pyrazin-2-yl-amine

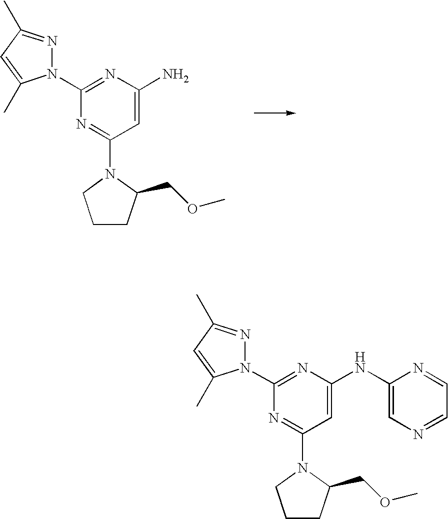

A mixture of Compound 1-96 (300 mg, 1.0 mmol, 1 eq.), (+/−)-2,2′-Bis(diphenylphosphino)-1,1′-binaphthalene (187 mg, 0.3 mmol, 0.3 eq.), palladium(II) acetate (67 mg, 0.3 mmol, 0.3 eq.), cesium carbonate (490 mg, 1.5 mmol, 1.5 eq.), and toluene (10 ml) was sparged with nitrogen gas for 10 min. 2-chloropyrazine (148 mg, 1.3 mmol, 1.3 eq.) was added, then the mixture was stirred and heated at 100° C. for 21 hr. Water was added, then the mixture was extracted with ethyl acetate. The combined extracts were dried over sodium sulfate, filtered, and concentrated. The residue was taken up in 50 ml of 1:1 dichloromethane/methanol and filtered to remove a white solid, which was discarded. The filtrate was concentrated and chromatographed on silica gel (5% methanol in dichloromethane eluant) to provide a yellow solid. Trituration (1:2 hexanes/ethyl acetate) gave the title compound (50 mg) as an off-white solid. LCMS (Method 1) m/z 381.0 [MH+], Tr=5.80 min

The compound of Table 1D was prepared by reacting compound 1-96 with the appropriate chloro representing the R³ substituent.

TABLE 1D

| | | | | | Reten | |
|---|---|---|---|---|---|---|
| Cmpd | | | | MS | time | HPLC |
| No. | Reactant | R³ | MW | ION | (min) | Method |
| 1-121 | 1-96 | 2-pyrazine | 380.4 | 381.0 | 5.8 | 1 |

The compounds of Table 1E are made by reacting the appropriate intermediate described above with the appropriate chloro, bromo, or iodo-substituted heterocycle representing the R³ substituent.

TABLE 1E

| Cmpd | Inter- | |
|---|---|---|
| No. | mediate | R³ |
| 1-206 | 1-96 | Thiazol-2-yl |
| 1-207 | 1-96 | 3,6-dimethylpyrazin-2-yl |
| 1-208 | 1-96 | Pyridine-2-yl |
| 1-209 | 1-96 | 6-methoxypyridazin-3-yl |
| 1-210 | 1-96 | Pyrimidin-2-yl |
| 1-211 | 1-96 | 3-cyanopyridin-2-yl |
| 1-212 | 1-96 | 4,6-dimethylpyrimidin-2-yl |
| 1-213 | 1-96 | 6-methylpyridin-2-yl |
| 1-214 | 1-96 | 6-methylpyridazin-3-yl |
| 1-215 | 1-96 | 4-methylpyridin-2-yl |
| 1-215 | 1-96 | 5-cyanopyridin-2-yl |
| 1-217 | 1-96 | 6-methoxypyridin-2-yl |
| 1-218 | 1-96 | 4-cyanopyridin-2-yl |
| 1-219 | 1-96 | 5-methylpyridin-2-yl |
| 1-220 | 1-96 | 3-cyanopyrazin-2-yl |
| 1-221 | 1-96 | 5-fluoropyridin-2-yl |
| 1-222 | 1-96 | 5-cyanopyridin-2-yl |
| 1-223 | 1-96 | 3-methoxypyridin-2-yl |
| 1-224 | 1-96 | 3-methylpyridin-2-yl |
| 1-225 | 1-96 | 1-methyl-1H-imidazol-4-yl |
| 1-226 | 1-96 | 4-methoxypyridin-2-yl |
| 1-227 | 1-96 | 1-methyl-1H-imidazol-2-yl |
| 1-228 | 1-96 | 1,3,4-thiadiazol-2-yl |
| 1-229 | 1-96 | Thiazol-4-yl |
| 1-230 | 1-96 | 1,4-dimethyl-1H-imidazol-2-yl |

### Intermediate 15: N-(6-Chloro-2-methylsulfanyl-pyrimidin-4-yl)-O-methyl-hydroxylamine

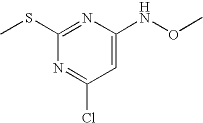

1 g (5.1 mmol, 1 eq) of 4,6-dichloro-2-methylthiopyrimidine, 0.43 g (5.1 mmol, 1 eq.) of methoxyamine hydrochloride and 1.8 mL diisopropylamine (10.2 mmol, 2 eq.) were heated in 20 mL dimethylformamide at 50° C. in a sealed vial overnight. After evaporation of solvents, the residue was purified by liquid chromatography on silica gel with hexanes and a gradient of ethyl acetate (0-20%) as eluent to yield 0.7 g (67% yield) of product. LCMS (Method 3) m/z 205.8 [MH+], Tr=2.52 min

### Intermediate 16: N-[6-Chloro-2-(3,5-dimethyl-pyrazol-1-yl)-pyrimidin-4-yl]-O-methyl-hydroxylamine

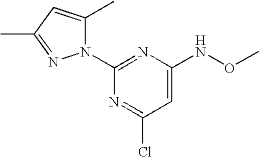

0.5 g (2.4 mmol, 1 eq.) of intermediate 15 was dissolved in THF/MeOH/water (3/10/10). 2.9 g (4.8 mmol, 2 eq.) of OXONE® was added and the mixture stirred at room temperature. After 3 hours, the reaction was complete. Water (10 mL) and ethyl acetate (50 mL) were added. The organic layer was separated and the aqueous layer extracted with ethyl acetate (2×50 mL). The organic layers were combined, dried over magnesium sulfate and evaporated to give the N-[6-Chloro-2-(3,5-dimethyl-pyrazol-1-yl)-pyrimidin-4-yl]-O-methyl-hydroxylamine as an oil. It was dissolved in 5 mL of ethanol and 0.2 mL (6.2 mmol, 2.6 eq.) of anhydrous hydrazine was added. After 2 hours of stirring at room temperature 0.3 mL (3.0 mmol, 1.2 eq.) of 2,4-pentanedione was added and the solution heated overnight at 50° C. After return to room temperature, solvents were evaporated, dichloromethane (200 mL) was added and washed with water (50 mL). The organic layer was dried over magnesium sulfate, filtered, and concentrated. The crude product was purified by flash chromatography on silica gel using 95:5 dichloromethane/methanol as eluent system. 0.5 g (40% yield) of Intermediate 14 was obtained as a yellow solid. LCMS (Method 3) m/z 253.8 [MH+], Tr=2.52 min

### Compound 1-122: N-[2-(3,5-Dimethyl-pyrazol-1-yl)-6-((R)-2-methoxymethyl-pyrrolidin-1-yl)-pyrimidin-4-yl]-O-methyl-hydroxylamine

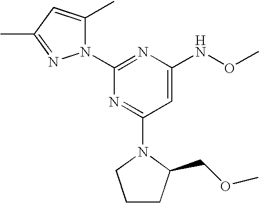

50 mg (0.2 mmol, 1 eq.) of Intermediate 16 and 50 mg (0.4 mmol, 2 eq.) of (R)-2-methoxynethylpyrrolidine were heated in 1 mL dioxane overnight at 80° C. in a sealed vial. After cooling down and evaporation of solvents, the residue was dissolved in DCM and purified by PTLC on silica gel with a mixture of dichloromethane/acetone in a ratio of 60/40 and with 1% ammonium hydroxide. LCMS (Method 1) m/z 332.9 [MH+], Tr=4.92 min

TABLE 1F

| | | | | | Reten | |
|---|---|---|---|---|---|---|
| Cmpd | | | | MS | time | HPLC |
| No. | Intermediate | R² | MW | ION | (min) | Method |
| 1-122 | 16 | (R)-2- | 332.4 | 332.9 | 4.9 | 1 |
| | | methoxymethyl- | | | | |
| | | pyrrolidin-1-yl | | | | |

### Intermediate 17: 2-Furancarboxamidine (HCl)

To a solution of sodium methoxide (5.55 mmol) in methanol (50 mL) was added 2-furonitrile (5.0 g, 53.2 mmol, 1 eq.). The mixture was stirred at room temperature for 3 hours. To the resulting solution was slowly added ammonium chloride (3.14 g, 58.7 mmol, 1.1 eq.) and the mixture was stirred at room temperature for 68 hours. The resulting suspension was filtered and the solvent removed under reduced pressure. The solid obtained was washed with ethyl ether (3×25 mL) to give 7.5 g (96% yield) of 2-furancarboxamidine (HCl).

6 (200 MHz, DMSO-d₆): 6.88-6.86 (m, 1H); 7.89 (d, J=3.8 Hz, 1H); 8.19 (s, 1H); 9.22 (s, 3H).

### Intermediate 18: 2-(2-Furyl)pyrimidine-4,6-diol

To a solution of sodium ethoxide (0.191 mol) in ethanol (90 mL) was slowly added furancarboxamidine.HCl 17 (5.6 g, 38.2 mmol, 1 eq.). The mixture was stirred at room temperature for 30 minutes and then, diethyl malonate (4.87 g, 30.4 mmol, 0.8 eq.) was added. The suspension was refluxed for 32 hours. The solvent was removed under reduced pressure, the residue was suspended in water (100 mL) and acidified to pH=6 with 5N hydrochloric acid. The resulting solid was filtered and washed with water (50 mL), ethanol/ethyl ether (4:1, 25 mL), ethyl ether (2×25 mL). 2-(2-Furyl)pyrimidine-4,6-diol was obtained (4.2 g, 78%) as a pale yellow solid.

δ (300 MHz, DMSO-d₆): 5.00 (s, 1H); 6.60-6.70 (m, 1H); 7.40 (d, J=3.4 Hz, 1H); 7.80 (s, 1H).

### Intermediate 19: 4,6-Dichloro-2-(2-furyl)pyrimidine

A suspension of Intermediate 18 (3.0 g, 16.8 mmol, 1 eq.) and N,N-diisopropylethylamine (3.85 g, 29.8 mmol, 1.8 eq.) in phosphorous oxychloride (17 mL) was refluxed for 3 hours. The solvent was removed under pressure and methylene chloride (50 mL) and ice were slowly added. The organic layer was washed with water (2×25 mL), saturated solution of sodium bicarbonate (2×25 mL), brine, and dried (Na₂SO₄). The solvent was removed under reduced pressure to give 4,6-dichloro-2-(2-furyl)pyrimidine (3.15 g, 87%) as a grey solid.

δ (300 MHz, CDCl₃): 6.63-6.61 (m, 1H); 7.22 (s, 1H); 7.46 (d, J=3.4 Hz, 1H); 7.68 (s, 1H).

### Intermediate 20: 6-Chloro-2-(2-furyl)pyrimidin-4-amine

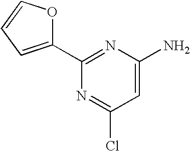

A suspension of Intermediate 19 (2.0 g, 9.3 mmol) in methanol (14 mL) and 30% ammonium hydroxide (27 mL) was heated in a pressure reactor for 20 hours. The solvent was partially removed under reduced pressure. The resulting solid was filtered, washed with water (25 mL), ethyl ether (25 mL), and dried. 6-Chloro-2-(2-furyl)pyrimidin-4-amine was obtained (1.48 g, 76%) as an off-white solid.

δ (400 MHz, CDCl₃): 5.21 (bs, 2H); 6.31 (s, 1H); 6.54 (m, 1H); 7.28 (d, J1=3.7 Hz, 1H); 7.58 (s, 1H).

### Intermediate 21: 6-Chloro-2-(5-methyl-furan-2-yl)-pyrimidin-4-ylamine

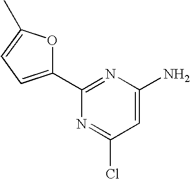

The title compound was obtained starting from 5-methyl-2-furonitrile by the procedure described in Intermediate 20.

### Intermediate 22: 6-Chloro-2-thiophen-2-yl-pyrimidin-4-ylamine

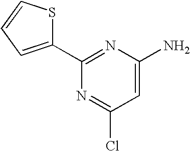

The title compound was obtained starting from thiophene-2-carbonitrile by the procedure described in Intermediate 20.

### Intermediate 23: 6-Chloro-2-thiazol-2-yl-pyrimidin-4-ylamine

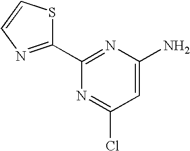

The title compound was obtained starting from thiaozol-2-carbonitrile by the procedure described in Intermediate 20.

### Intermediate 24: 6-Chloro-2-pyridin-2-yl-pyrimidin-4-ylamine

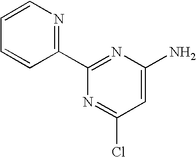

The title compound was obtained starting from 2-cyanopyridine by the procedure described in Intermediate 20.

### Intermediate 25: N-(6-Chloro-2-furan-2-yl-pyrimidin-4-yl)-acetamide

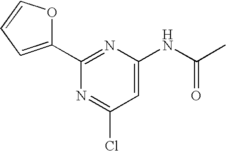

Intermediate 25 was obtained by acylating Intermediate 20 according to the procedure of Intermediate 5.

### Intermediate 26: N-[6-Chloro-2-(5-methyl-furan-2-yl)-pyrimidin-4-yl]acetamide

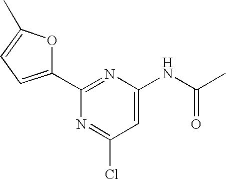

Intermediate 26 was obtained by acylating Intermediate 21 according to the procedure of Intermediate 5.

### Intermediate 27: N-(6-Chloro-2-thiophen-2-yl-pyrimidin-4-yl)-acetamide

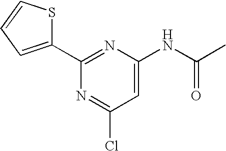

Intermediate 27 was obtained by acylating Intermediate 22 according to the procedure of Intermediate 5.

### Intermediate 28: N-(6-Chloro-2-thiazol-2-yl-pyrimidin-4-yl)-acetamide

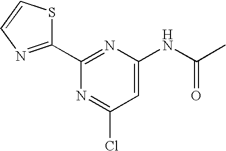

Intermediate 28 was obtained by acylating Intermediate 23 according to the procedure of Intermediate 5.

### Intermediate 29: N-(6-Chloro-2-pyridin-2-yl-pyrimidin-4-yl)-acetamide

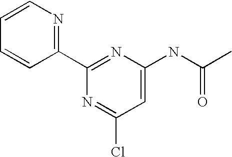

Intermediate 29 was obtained by acylating Intermediate 24 according to the procedure of Intermediate 5.

### Intermediate 30: N-[6-Chloro-2-(5-methyl-furan-2-yl)-pyrimidin-4-yl]-2-(4-methoxy-phenyl)-acetamide

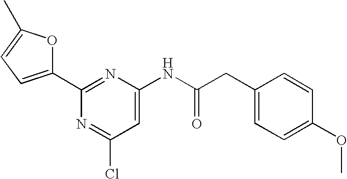

Intermediate 30 was obtained by acylating Intermediate 21 according to the procedure of Intermediate 11 using p-methoxyphenylacetyl chloride instead of chloroacetyl chloride.

The compounds of Table 2A were prepared by reacting the appropriate intermediate described above with the appropriate amine representing the R² substituent:

TABLE 2A

| | | | | | Reten | |
|---|---|---|---|---|---|---|
| Cmpd | | | | MS | time | HPLC |
| No. | Intermediate | R² | MW | ION | (min) | Method |
| 2-1 | 25 | (R)-2- | 316.4 | 317.7 | 7.7 | 2 |
| | | methoxymethyl- | | | | |
| | | pyrrolidin-1-yl | | | | |
| 2-2 | 26 | (R)-2- | 330.4 | 331.4 | 8.1 | 2 |
| | | methoxymethyl- | | | | |
| | | pyrrolidin-1-yl | | | | |
| 2-3 | 26 | (R)-2- | 330.4 | 331.4 | 8.1 | 2 |
| | | methoxymethyl- | | | | |
| | | pyrrolidin-1-yl | | | | |
| 2-4 | 26 | (S)-2- | 316.4 | 317.1 | 6.5 | 2 |
| | | methoxymethyl- | | | | |
| | | pyrrolidin-1-yl | | | | |
| 2-5 | 26 | bis-(2-methoxy- | 348.4 | 349.4 | 7.5 | 2 |
| | | ethyl)-amino | | | | |
| 2-6 | 26 | methyl-(3- | 380.4 | 380.8 | 8.2 | 2 |
| | | phenyl- | | | | |
| | | butyl)-amino | | | | |
| 2-7 | 26 | piperidin-1-yl | 300.4 | 301.1 | 8.6 | 2 |
| 2-8 | 26 | (2-hydroxy- | 290.3 | 290.8 | 5.8 | 2 |
| | | ethyl)- | | | | |
| | | methyl-amino | | | | |
| 2-9 | 26 | ethyl-pyridin-4- | 351.4 | 351.9 | 7.3 | 2 |
| | | ylmethyl-amino | | | | |
| 2-10 | 26 | 2,6-dimethyl- | 328.4 | 329.1 | 9.8 | 2 |
| | | piperidin-1-yl | | | | |
| 2-11 | 26 | [1,3]dioxolan-2- | 332.4 | 333.2 | 7.1 | 2 |
| | | ylmethyl- | | | | |
| | | methyl-amino | | | | |
| 2-12 | 26 | dimethylamino | 260.3 | 260.9 | 7.2 | 2 |
| 2-13 | 26 | azetidin-1-yl | 272.3 | 273.1 | 6.8 | 2 |
| 2-14 | 26 | 2-propyl- | 328.4 | 329.4 | 9.9 | 2 |
| | | pyrrolidin-1-yl | | | | |
| 2-15 | 30 | dimethylamino | 366.4 | 367.0 | 5.7 | 1 |
| 2-16 | 26 | 3-hydroxy- | 316.4 | 316.5 | 6.2 | 2 |
| | | piperidin-1-yl | | | | |
| 2-17 | 26 | 4- | 344.4 | 345.3 | 8.2 | 2 |
| | | methoxymethyl- | | | | |
| | | piperidin-1-yl | | | | |
| 2-18 | 26 | (S)-2- | 330.4 | 331.3 | 8.1 | 2 |
| | | methoxymethyl- | | | | |
| | | pyrrolidin-1-yl | | | | |
| 2-19 | 26 | morpholin-4-yl | 302.3 | 302.9 | 6.8 | 2 |
| 2-20 | 21 | (S)-2- | 274.3 | 275.1 | 5.9 | 2 |
| | | hydroxymethyl- | | | | |
| | | pyrrolidin-1-yl | | | | |
| 2-21 | 21 | (S)-2- | 288.3 | 288.8 | 7.4 | 2 |
| | | methoxymethyl- | | | | |
| | | pyrrolidin-1-yl | | | | |
| 2-22 | 26 | (S)-3- | 329.4 | 329.5 | 6.5 | 2 |
| | | Dimethylamino- | | | | |
| | | pyrrolidin-1-yl | | | | |
| 2-23 | 26 | 2-Ethoxymethyl- | 344.4 | 344.7 | 8.6 | 2 |
| | | pyrrolidin-1-yl | | | | |
| 2-24 | 26 | 4-methoxy- | 330.4 | 330.9 | 7.5 | 2 |
| | | piperidin-1-yl | | | | |
| 2-25 | 28 | (S)-2- | 333.4 | 334.0 | 4.6 | 1 |
| | | methoxymethyl- | | | | |
| | | pyrrolidin-1-yl | | | | |
| 2-26 | 28 | (R)-2- | 333.4 | 334.0 | 4.6 | 1 |
| | | methoxymethyl- | | | | |
| | | pyrrolidin-1-yl | | | | |
| 2-27 | 27 | (S)-2- | 332.4 | 333.5 | 8.8 | 1 |
| | | methoxymethyl- | | | | |
| | | pyrrolidin-1-yl | | | | |
| 2-28 | 29 | (R)-2- | 327.4 | 327.7 | 6.7 | 1 |
| | | methoxymethyl- | | | | |
| | | pyrrolidin-1-yl | | | | |
| 2-29 | 27 | (R)-2- | 332.4 | 332.9 | 8.7 | 1 |
| | | methoxymethyl- | | | | |
| | | pyrrolidin-1-yl | | | | |
| 2-30 | 29 | (S)-2- | 327.4 | 328.1 | 6.7 | 1 |
| | | methoxymethyl- | | | | |
| | | pyrrolidin-1-yl | | | | |
| 2-31 | 29 | methoxyamino | 259.3 | 260.1 | 5.1 | 1 |

The compounds of Table 2B are prepared by reacting the appropriate intermediate described above with the appropriate amine representing the R².

TABLE 2B

| Cmpd No. | Intermediate | R² |
|---|---|---|
| 2-200 | 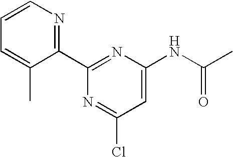 | (R)-2- methoxymethyl- pyrrolidin-1-yl |
| 2-201 | 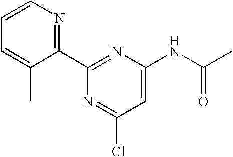 | (S)-2- methoxymethyl- pyrrolidin-1-yl |

### Intermediate 31: 6-Chloro-2-(3,5-dimethyl-pyrazol-1-yl)-pyrimidin-4-ylamine

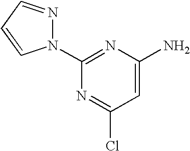

6-amino-2,4-dichloropyrimidine (25.0 g, 164 mmol, 1 eg.), pyrazole (15.5 g, 228 mmol, 1.5 eq.) and cesium carbonate (16.4 g, 228 mmol, 1.5 eq.) were heated at reflux in dioxane (150 mL) for 3 days. The reaction was allowed to cool down to room temperature, filtered over celite. The celite was washed with dioxane (300 mL) and the filtrate was concentrated under vacuum. The residue was slurried with dichloromethane for 16 hours and filtered to give 8.1 g of intermediate 24 as an off white solid. The operation was repeated with the mother liquor to get a second crop (3.6 g) (39% overall yield). LCMS (Method 3) m/z 196.0 [MH+], Tr=1.99 min

### Intermediate 32: N-(6-Chloro-2-pyrazol-1-yl-pyrimidin-4-yl)-acetamide

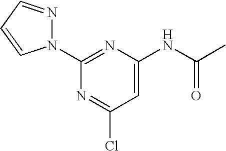

Intermediates 31 was acylated by the procedure described for intermediate 5 to yield to Intermediate 32 in similar yields. LCMS (Method 3) m/z 238.2 [MH+], Tr=2.28 min

### Intermediate 33: N-[6-Chloro-2-(pyrazol-1-yl)-pyrimidin-4-yl]-3-methyl-butyramide

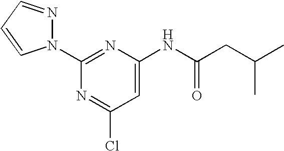

Intermediate 33 was prepared by reacting Intermediate 31 with isobutyryl chloride in a similar way as for Intermediate 10. The residue was purified by liquid chromatography using a mixture of 1 to 1 ethyl acetate/hexanes to afford a white solid in similar yields. LCMS (Method 3) m/z 280.0 [MH+], Tr=2.69 min

### Intermediate 34: 2-Chloro-N-[6-chloro-2-(pyrazol-1-yl)-pyrimidin-4-yl]acetamide

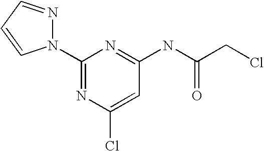

Intermediate 34 was prepared by reacting Intermediate 31 with chloroacetyl chloride in a similar way as for Intermediate 11.

Intermediate 35: N-[6-Chloro-2-(pyrazol-1-yl)-pyrimidin-4-yl]-2-(4-methyl-piperazin-1-yl)-acetamide

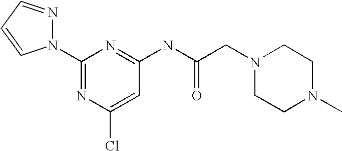

Intermediate 35 was prepared by reacting Intermediate 34 with N-methyl piperidine in a similar way as for Intermediate 12.

The compounds of Table 3 were prepared by reacting the appropriate intermediate described above with the appropriate amine representing the R² substituent:

TABLE 3

| Cmpd | | | | MS | Reten time | HPLC |
|---|---|---|---|---|---|---|
| No. | Intermediate | R² | MW | ION | (min) | Method |
| 3-1 | 33 | (R)-2-methoxymethyl- | 358.4 | 359.2 | 0.8 | 1 |
| | | pyrrolidin-1-yl | | | | |
| 3-2 | 33 | 3,5-dimethylpiperidin-1- | 356.5 | 357.2 | 0.9 | 1 |
| | | yl | | | | |
| 3-3 | 32 | (R)-2-methoxymethyl- | 316.4 | 317.4 | 6.7 | 1 |
| | | pyrrolidin-1-yl | | | | |
| 3-4 | 33 | 3,4-Dihydro-1H- | 376.5 | 377.1 | 1.0 | 1 |
| | | isoquinolin-2-yl | | | | |
| 3-5 | 35 | 6-morpholin-4-yl | 386.5 | 387.9 | 5.8 | 2 |

It will be appreciated that, although specific embodiments of the invention have been described herein for purposes of illustration, various modifications may be made without departing from the spirit and scope of the invention. Accordingly, the invention is not limited except as by the appended claims.

What is claimed is:

1. A compound of formula (I) 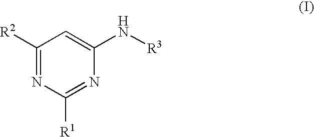
or a pharmaceutically acceptable salt, ester, solvate or stereoisomer thereof, wherein:
R¹ is a heterocycle optionally substituted by one or more members selected from the group of lower alkyl, lower alkoxy, halogen and cyano;
R² is NR⁴R⁵ or a heterocycle, wherein the heterocycle is substituted by 0 to 4 R⁴ groups;
R³ is H, R⁶, OR⁶, COR⁶, CONR⁶R⁷, COOR⁶, or a heteroaryl having at least one nitrogen wherein the heteroaryl is optionally substituted by 0 to 4 R⁴;
R⁴ is at each occurrence selected from the group of lower alkyl, lower alkoxy, alkoxyalkyl, oxo, cyano, halogen, hydroxy, —C(O)-alkyl, lower alkenyl, aryl, heteroaryl, arylalkyl, heteroarylalkyl, heterocycle and heterocyclealkyl, wherein the lower alkyl, lower alkoxy, alkoxyalkyl, aryl, heteroaryl, arylalkyl, heteroarylalkyl, heterocycle and heterocyclealkyl groups are optionally substituted by one or more lower alkyl, halogen, lower alkoxy, hydroxyl, cyano, aryl and —C(O)-alkyl;
R⁵ is at each occurrence selected from the group of hydrogen, lower alkyl, lower alkoxy and alkoxyalkyl;
R⁶ is lower alkyl, arylalkyl, heteroaryl or heterocyclealkyl, wherein the lower alkyl, arylalkyl, heteroaryl and heterocyclealkyl groups are optionally substituted by one or more members selected from the group of lower alkyl, lower alkoxy, hydroxyl, oxo, halogen, amino, alkylamino and dialkylamino; and
R⁷ is hydrogen or lower alkyl, wherein the lower alkyl group is optionally substituted by one or more members selected from the group of alkoxy, hydroxyl, oxo, halogen, amino, alkylamino and dialkylamino.

2. A compound according to claim 1, wherein R¹ is selected from the group of pyrazolyl, triazolyl, furanyl, thiazolyl and pyridinyl, wherein the pyrazolyl, triazolyl, furanyl, thiazolyl and pyridinyl groups are optionally substituted by one or more member selected from the group of lower alkyl and halogen.

3. A compound according to claim 2, wherein R¹ is pyrazolyl optionally substituted by two lower alkyl groups or furanyl optionally substituted by one lower alkyl group.

4. A compound according to claim 3, wherein R¹ is selected from the group of pyrazol-1-yl, 3,5-dimethyl-pyrazol-1-yl, furan-2-yl and 5-methyl-furan-2-yl.

5. A compound according to claim 4, wherein R¹ is 3,5-dimethyl-pyrazol-1-yl or 5-methyl-furan-2-yl.

6. A compound according to claim 1, wherein R² is a heterocycle selected from the group of pyrrolidinyl, piperidinyl, indolyl, isoindolyl, tetrahydroquinolyl, lactonyl and piperazinyl, wherein the pyrrolidinyl, piperidinyl, indolyl, isoindolyl, tetrahydroquinolyl, lactonyl, lactamyl, tetrahydropyridinyl and piperazinyl groups are optionally substituted by 0 to 4 R⁴ groups.

7. A compound according to claim 1, wherein R² is a heterocycle selected from the group of pyrrolidinyl, piperidinyl, indolyl, isoindolyl, tetrahydroquinolyl, lactonyl and piperazinyl, wherein the pyrrolidinyl, piperidinyl, indolyl, isoindolyl, tetrahydroquinolyl, lactonyl, lactamyl, tetrahydropyridinyl and piperazinyl groups are optionally substituted by 0 to 2 R⁴ groups.

8. A compound according to claim 1, wherein R² is pyrrolidinyl, piperidinyl, indolyl or isoindolyl, wherein the pyrrolidinyl, piperidinyl, indolyl or isoindolyl are optionally substituted by 0 to 2 R⁴ groups.

9. A compound according to claim 1, wherein R² is pyrrolidinyl, piperidinyl, indolyl or isoindolyl, wherein the pyrrolidinyl, piperidinyl, indolyl or isoindolyl are optionally substituted by 1 to 2 R⁴ groups.

10. A compound according to claim 1, wherein R³ is COR^{E}.

11. A compound according to claim 10, wherein R⁶ is lower alkyl.

12. A compound according to claim 11, wherein R⁶ is methyl, ethyl or isopropyl.

13. A compound according to claim 12, wherein R⁶ is methyl.

14. A pharmaceutical composition comprising a compound according to claim 1 and a pharmaceutically acceptable carrier or diluent.

15. A pharmaceutical composition comprising a compound according to claim 3 and a pharmaceutically acceptable carrier or diluent.

16. A pharmaceutical composition comprising a compound according to claim 7, and a pharmaceutically acceptable carrier or diluent.

17. A pharmaceutical composition comprising a compound according to claim 11, and a pharmaceutically acceptable carrier or diluent.

18. A pharmaceutical composition comprising a compound according to claim 13, and a pharmaceutically acceptable carrier or diluent.

19. A method for treating a subject having a condition susceptible to amelioration by antagonism of A_{2A} adenosine receptor comprising administering to said subject a pharmaceutical composition according to claim 1.

20. A method according to claim 19 wherein the condition is ischemia, supraventricular arrhythmias, acute renal failure, myocardial reperfusion injury, autoimmune disease, inflammatory bowel diseases, asthma, diabetes mellitus, obesity, Parkinson's disease, Huntington's disease, dystonia or dyskinesia.

21. A method according to claim 20 wherein the condition is ischemia, supraventricular arrhythmias, Parkinson's disease, Huntington's disease, dystonia or dyskinesia.

22. A method according to claim 21, wherein the condition is Parkinson's disease.
